(19) United States
(12) Patent Application Publication
(10) Pub. No.: US 2024/0399142 A1
(43) Pub. Date: Dec. 5, 2024

(54) Methods Of Using Alternating Electric Fields And Checkpoint Inhibitors

(71) Applicant: NOVOCURE GMBH, Root D4 (CH)

(72) Inventor: Uri Weinberg, Haifa (IL)

(21) Appl. No.: 18/680,762

(22) Filed: May 31, 2024

Related U.S. Application Data
(60) Provisional application No. 63/505,616, filed on Jun. 1, 2023.
(60) Provisional application No. 63/586,730, filed on Sep. 29, 2023.

(51) Int. Cl. A61N 1/32; A61K 31/337; C07K 16/28

(52) U.S. Cl. CPC A61N 1/32; A61K 31/337; C07K 16/2818; C07K 16/2827; C07K 2317/21; C07K 2317/24

(57) ABSTRACT

Disclosed are methods of increasing survival of a subject having cancer comprising applying an alternating electric field to a target site of the subject for a period of time, the alternating electric field having a frequency and field strength, wherein the target site comprises one or more cancer cells, and administering a therapeutically effective amount of a checkpoint inhibitor to the subject. Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a checkpoint inhibitor to the subject. In some aspects, the checkpoint inhibitor is Nivolumab, Pembrolizumab, or Atezolizumab. Also disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject. Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject. In some aspects, the checkpoint inhibitor is ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio).

### CROSS-REFERENCE TO RELATED APPLICATIONS

This application claims the benefit of U.S. Provisional Patent Application Nos. 63/505,616, filed Jun. 1, 2023, and 63/586,730, filed Sep. 29, 2023, each of which is incorporated by reference herein in its entirety.

### BACKGROUND

Worldwide, lung cancer is the leading cause of cancer death with an estimated 1.8 million deaths in 2020. In the United States, there are over 230,000 new cases of lung cancer and 130,000 deaths annually. Approximately 84% of patients with lung cancer have NSCLC and about 60% of them have distant metastases by the time of diagnosis (stage IV).

Metastatic non-small cell lung cancer (NSCLC) remains uncurable, despite the introduction of immune checkpoint inhibitors.

Checkpoint inhibitors are a type of immunotherapy, approved for the treatment of different cancers such as melanoma, cervical, colorectal and lung cancer. These drugs block different checkpoint proteins. One might also hear them named after these checkpoint proteins—for example, CTLA-4 inhibitors, PD-1 inhibitors and PD-L1 inhibitors. NCCN guidelines don't recommend using PD-1/PD-L1 inhibitor if progression has occurred on PD-1/PD-L1 inhibitor. The standard of care therapies for patients progressed after PD-1/PD-L1 inhibitor are mainly different chemotherapies or combination of docetaxel and ramucirumab. Per the ESMO guidelines, combination of docetaxel with nintedanib is an additional optional treatment for patients with adenocarcinoma progressing after chemotherapy and PD-1/PD-L1.

Platinum-based therapy remains part of standard of care, either in combination with or after a first line immune checkpoint inhibitor, or for patients who cannot tolerate an immune checkpoint inhibitor. However, once a patient has progressed on platinum, there are few treatments shown to extend survival; current approaches include other chemotherapy regimens (mainly docetaxel), with or without ramucirumab, or immune checkpoint inhibitor rechallenge.

Tumor Treating Fields (TTFields; for example, at 150 kHz) are electric fields that disrupt processes critical for cancer cell survival and tumor progression. TTFields therapy is delivered locoregionally and noninvasively to the tumor site by a portable medical device via two pairs of arrays placed on the patient's skin (FIG. 3). TTFields therapy is FDA-approved and CE marked for glioblastoma based on two randomized, phase 3 studies, as well as unresectable pleural mesothelioma. TTFields therapy is not associated with systemic toxicity; the most common device-related adverse events are manageable mild-to-moderate beneath-array skin irritation which occur due to contact with components of the device.

TTFields affect cancer cells by a unique multi-modal mechanism of action that includes impairing the DNA damage response and disrupting mitosis. In preclinical models of NSCLC, the subsequent induction of cellular stress and immunogenic cell death was shown to enhance the antitumor immune response. In addition, TTFields treatment amplified the preclinical effectiveness of immune checkpoint inhibitors or taxanes. TTFields have been associated with stimulation of the STING pathway, which in turn is associated with anti-tumor effects.

STING has also been implicated in promoting tumour burden and worse disease outcomes in models of cancer. Whilst transient activation appears favorable, adverse effects seem to rely on the stimulation of a chronic inflammatory program that promotes an immune-suppressive tumour environment. STING ligands can also induce the expression of inhibitory molecules, which counteracts the tumour-suppressive effects of type I interferons. The pro-apoptotic effects of STING on T cells may also contribute to limited tumour cell clearance. In the context of more advanced disease, a STING-mediated inflammatory retrenchment may allow aggressive cancer cells to adapt, and overcome and finally to further thrive and propagate. Chronic STING signaling may therefore represent a key problem that must be taken into consideration for tumour therapies targeting this pathway (Decout, A., Katz, J. D., Venkatraman, S. et al. The cGAS-STING pathway as a therapeutic target in inflammatory diseases. Nat Rev Immunol 21, 548-569 (2021)).

The majority of NSCLC patients eventually experience disease progression, become intolerant of symptomatology, or develop resistance to available treatments, and OS is low for patients with advanced disease. Therefore, there is a need for new treatments that can prolong survival in this patient population, without exacerbating treatment side effects, including quality of life.

### BRIEF SUMMARY

Disclosed are methods of increasing survival of a subject having cancer comprising applying an alternating electric field to a target site of the subject for a period of time, the alternating electric field having a frequency and field strength, wherein the target site comprises one or more cancer cells, and administering a therapeutically effective amount of a checkpoint inhibitor to the subject.

Disclosed are checkpoint inhibitors for use in methods of increasing survival of a subject having cancer comprising applying an alternating electric field to a target site of the subject for a period of time, the alternating electric field having a frequency and field strength, wherein the target site comprises one or more cancer cells, and administering a therapeutically effective amount of the checkpoint inhibitor to the subject.

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a checkpoint inhibitor to the subject.

Disclosed are checkpoint inhibitors for use in methods of treating a subject having cancer comprising applying an alternating electric field to a target site of the subject for a period of time, the alternating electric field having a frequency and field strength, wherein the target site comprises one or more cancer cells, and administering a therapeutically effective amount of the checkpoint inhibitor to the subject.

In some aspects, the checkpoint inhibitor is Nivolumab, Pembrolizumab, or Atezolizumab.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject.

Disclosed are checkpoint inhibitors for use in methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of the checkpoint inhibitor to the subject.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of an checkpoint inhibitor to the subject, wherein the non-small cell lung cancer in the subject progressed on or after the previous treatment with the checkpoint inhibitor.

Disclosed are checkpoint inhibitors for use in methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of the checkpoint inhibitor to the subject, wherein the non-small cell lung cancer in the subject progressed on or after the previous treatment with the checkpoint inhibitor.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the non-small cell lung cancer in the subject progressed on or after the previous treatment with the checkpoint inhibitor and the chemotherapy.

Disclosed are checkpoint inhibitors for use in methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of the checkpoint inhibitor to the subject, wherein the non-small cell lung cancer in the subject progressed on or after the previous treatment with the checkpoint inhibitor and the chemotherapy.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was not previously treated with a non-platinum-based chemotherapy or a platinum-based chemotherapy.

Disclosed are checkpoint inhibitors for use in methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of the checkpoint inhibitor to the subject, wherein the subject was not previously treated with a non-platinum-based chemotherapy or a platinum-based chemotherapy.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and was not previously treated with a platinum-based chemotherapy.

Disclosed are checkpoint inhibitors for use in methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of the checkpoint inhibitor to the subject, wherein the subject was previously treated with a checkpoint inhibitor and was not previously treated with a platinum-based chemotherapy.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject.

Disclosed are checkpoint inhibitors for use in methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of the checkpoint inhibitor to the subject.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the non-small cell lung cancer in the subject progressed on or after the previous treatment with the checkpoint inhibitor.

Disclosed are checkpoint inhibitors for use in methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of the checkpoint inhibitor to the subject, wherein the non-small cell lung cancer in the subject progressed on or after the previous treatment with the checkpoint inhibitor.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the non-small cell lung cancer in the subject progressed on or after the previous treatment with the checkpoint inhibitor and the chemotherapy.

Disclosed are checkpoint inhibitors for use in methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of the checkpoint inhibitor to the subject, wherein the non-small cell lung cancer in the subject progressed on or after the previous treatment with the checkpoint inhibitor and the chemotherapy.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and was not previously treated with a non-platinum-based chemotherapy or a platinum-based chemotherapy.

Disclosed are checkpoint inhibitors for use in methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with the checkpoint inhibitor and was not previously treated with a non-platinum-based chemotherapy or a platinum-based chemotherapy.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and was not previously treated with a platinum-based chemotherapy.

Disclosed are checkpoint inhibitors for use in methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and was not previously treated with a platinum-based chemotherapy.

In some aspects, the checkpoint inhibitor is ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio).

Disclosed are taxanes for use methods of increasing survival of a subject having cancer comprising: applying an alternating electric field to a target site of the subject for a period of time, the alternating electric field having a frequency and field strength, wherein the target site comprises one or more cancer cells, and administering a therapeutically effective amount of the taxane to the subject.

Disclosed are taxanes for use methods of treating a subject having cancer comprising: applying an alternating electric field to a target site of the subject for a period of time, the alternating electric field having a frequency and field strength, wherein the target site comprises one or more cancer cells, and administering a therapeutically effective amount of the taxane to the subject.

Disclosed are methods of increasing survival of a subject having cancer comprising: applying an alternating electric field to a target site of the subject for a period of time, the alternating electric field having a frequency and field strength, wherein the target site comprises one or more cancer cells, and administering a therapeutically effective amount of a taxane to the subject.

Disclosed are methods of treating a subject having cancer comprising: applying an alternating electric field to a target site of the subject for a period of time, the alternating electric field having a frequency and field strength, wherein the target site comprises one or more cancer cells, and administering a therapeutically effective amount of a taxane to the subject.

In some aspects, the taxane is docetaxel, paclitaxel, cabazitaxel, abraxane, or a combination thereof.

Additional advantages of the disclosed methods and compositions will be set forth in part in the description which follows, and in part will be understood from the description, or may be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows an example of overall survival in the wverall population (Kaplan-Meier estimate of overall survival).

FIGS. 2A and 2B show an example of overall survival in the treatment subgroups (Kaplan-Meier estimates of overall survival). FIG. 2A shows overall survival in the checkpoint inhibitor subgroup. FIG. 2B shows overall survival in the docetaxel subgroup.

FIGS. 3A-3D show the tumor treating fields (TTFields) medical device—an example of devices that generate and deliver TTFields. FIGS. 3A & 3B show components of the wearable first-generation (NovoTTF-100L) and second-generation (NovoTTF-200T) devices that generate and deliver TTFields. The lighter weight (1.2 kg vs 2.7 kg) NovoTTF-200T device was introduced during the course of patient enrollment, and patients were eligible to use either device. FIG. 3C shows an image showing an actor (not a patient) with an example array layout to target NSCLC. FIG. 3D is an image showing an x-ray/CT/PET scan of the thorax, with tumor location highlighted, overlaid with a projection of field simulation.

FIG. 4 shows a study design and protocol amendment. The protocol originally assigned patients to the NovoTTF-100L system. This was updated to allow use of the smaller and lighter second-generation NovoTTF-200T system. Following pre-specified annual review of safety and overall survival data by the Data Monitoring Committee (31 March, 2021), the committee recommended that study endpoints could be analyzed using a reduced sample size of 276 patients and 12 month follow-up. CT, computed tomography; FDA, US Food and Drug Administration; ICI, immune check point inhibitor, OS, overall survival; Q6W, every 6 weeks; R, randomization; TTFields, Tumor Treating Fields.

FIGS. 5A and 5B show overall survival in patients with squamous and non-squamous disease, where standard therapy refers to an immune checkpoint inhibitor (FIG. 5A) or docetaxel (FIG. 5B).

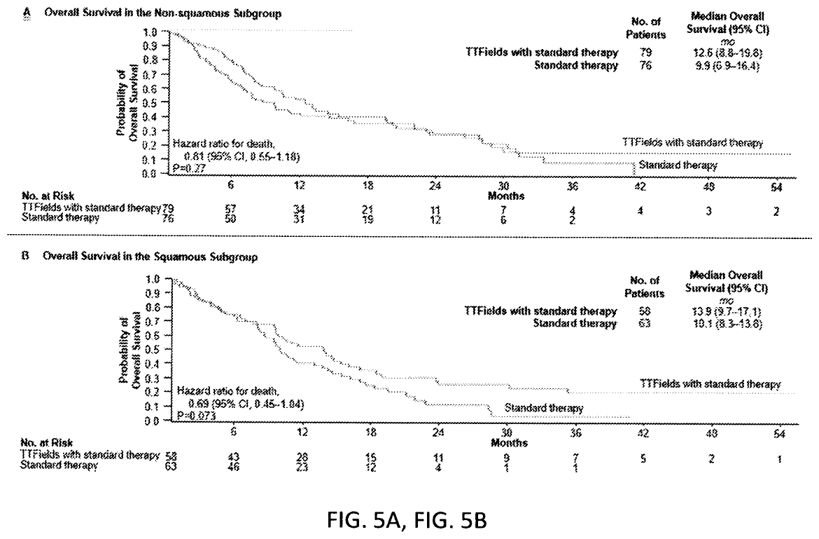

FIG. 6 shows progression-free survival in the overall population of patients, where standard therapy refers to an immune checkpoint inhibitor or docetaxel.

FIGS. 7A and 7B show progression-free survival in patients receiving an immune checkpoint inhibitor (FIG. 7A) or Docetaxel (FIG. 7B).

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a checkpoint inhibitor” includes a plurality of such inhibitors, reference to “the checkpoint inhibitor” is a reference to one or more inhibitors and equivalents thereof known to those skilled in the art, and so forth.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the lungs. In some aspects, a cell or population of cells that can be a target site or a target cell include, but are not limited to, a cancer cell (e.g., a lung cancer cell). In some aspects, a “target site” can be a tumor target site.

A “tumor target site” is a site or location within or present on a subject or patient that comprises or is adjacent to one or more non-small cell lung cancer cells, previously comprised one or more tumor cells, or is suspected of comprising one or more tumor cells. For example, a tumor target site can refer to a site or location within or present on a subject or patient that is prone to metastases (e.g. thorax). Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site). In some aspects, the alternating electrical field can be in a single direction or multiple directional. In some aspects, alternating electric fields can be delivered through two pairs of transducer arrays that generate perpendicular fields within the treated target region. For example, for the Optune™ system (an alternating electric fields delivery system) one pair of electrodes is located to the left and right (LR) of the target region, and the other pair of electrodes is located anterior and posterior (AP) to the target region. Cycling the field between these two directions (i.e., LR and AP) ensures that a maximal range of cell orientations is targeted. In some aspects, an alternating electric field can be referred to as Tumor Treating Field (TTField).

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and are described in U.S. Pat. No. 7,565,205, which is incorporated herein by reference in its entirety for its teaching of TTFields.

In-vivo and in-vitro studies show that the efficacy of alternating electric fields therapy increases as the intensity of the electrical field increases. Therefore, optimizing array placement on the area of a patient's tumor to increase the intensity in the desired region of the tumor can be performed with the Optune system. Array placement optimization may be performed by “rule of thumb” (e.g., placing the arrays on the tumor as close to the desired region of the target site (e.g., cancer cells) as possible), measurements describing the geometry of the patient's tumor, tumor dimensions. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target region as a function of the positions of the array and, in some aspects, take account for variations in the electrical property distributions within the same target region of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field and/or the therapeutically effective amount of a checkpoint inhibitor.

By “treat” is meant to administer or apply a therapeutic, such as alternating electric fields and/or a checkpoint inhibitor, to a subject, such as a human or other mammal (for example, an animal model), that has cancer or has an increased susceptibility for developing cancer, in order to prevent or delay a worsening of the effects of the cancer, or to partially or fully reverse the effects of the cancer (glioblastoma, ovarian, or lung metastatic carcinoma).

By “prevent” is meant to minimize the chance that a subject who has an increased susceptibility for developing cancer will develop cancer.

As used herein, the terms “administering” and “administration” refer to any method of providing a therapeutic, such as a checkpoint inhibitor to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing. Thus, in some aspects, exposing a cancer cell to alternating electrical fields means administering alternating electrical fields to the cancer cell.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

The word “or” as used herein means any one member of a particular list and also includes any combination of members of that list.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present methods and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field (TTFields). In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be 50 kHz, 100 kHz, 200 kHz, 300 kHz, 400 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the alternating electric field may be induced by an applied voltage of at least 50 V RMS. In some aspects, the alternating electric field can be induced by an applied voltage of at least 25-200 V RMS. In some aspects, the alternating electric field can be induced by an applied voltage of at least about 5, 10, 15, 20, 25, 50, 75, 100, 125, 150, 175, 200, 225, 250, 275 or 300 V RMS.

In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two-hour duration.

In some aspects, the exposure may last for at least 6 hours, at least 12 hours, at least 24 hours, at least 36 hours, at least 48 hours, or at least 72 hours or more. In some aspects, the exposure can be consecutive or cumulative. In some aspects, the consecutive exposure may last for at least 6 hours, at least 12 hours, at least 24 hours, at least 36 hours, at least 48 hours, or at least 72 hours or more. In some aspects, the cumulative exposure may last for at least 42 hours, at least 84 hours, at least 168 hours, at least 250 hours, at least 400 hours, at least 500 hours, at least 750 hours, or more. In some aspects, there can be a break in treatment and the alternating electric fields are applied at least 50%, 60%, 70%, or 80% of treatment time. For example, in some aspects, cumulative exposure can be for at least 12 hours in a period of 24 hours.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

In some aspects, the exposure may last for at least 6 hours, at least 12 hours, at least 24 hours, at least 36 hours, at least 48 hours, or at least 72 hours or more.

In addition, when the alternating electric field is applied to a subject, the period of time that the alternating electric field is applied may be measured in terms of a continuous period of time or a cumulative period of time. That is, the period of time that the alternating electric field is applied may include a single session (i.e., continuous application) as well as multiple sessions with minor breaks in between sessions (i.e., consecutive applications for a cumulative period). For example, even within a continuous application, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.). For example, the alternating electric field can be applied for at least 12 hours, 16 hours, or 18 hours cumulative each day for a week, a month, two months, three months, etc.

### C. Checkpoint Inhibitors

In some aspects, the terms “checkpoint inhibitors” and “immune checkpoint inhibitors” can be used interchangeably.

The methods disclosed herein comprise administering one or more checkpoint inhibitors to a subject. In some aspects, the checkpoint inhibitor can block CTLA-4 (cytotoxic T lymphocyte associated protein 4), PD-1 (programmed cell death protein 1) or PD-L1 (programmed cell death ligand 1).

In some aspects, the checkpoint inhibitor can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, Tremelimumab, Sintilimab (formerly IB1308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In some aspects, the methods pertain to a subject who was previously treated with a checkpoint inhibitor. In such embodiments the checkpoint inhibitor can be an inhibitor that blocks CTLA-4 (cytotoxic T lymphocyte associated protein 4), PD-1 (programmed cell death protein 1) or PD-L1 (programmed cell death ligand 1). In some aspects, the checkpoint inhibitor previously administered to the subject can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, tremelimumab, sintilimab (formerly IB1308; tyvyt), tislelizumab (formerly BGB-A317), toripalimab (formerly JS 001), spartalizumab (formerly PRD001); camrelizumab (formerly SHR1210), KN035, cosibelimab (formerly CK-301), CA-170, or BMS-986189, or a combination thereof.

In some aspects, the checkpoint inhibitor is a PD-1 inhibitor, PD-L1 inhibitor, or CTLA4 inhibitor. In some aspects, a PD-1 inhibitor can be, but is not limited to, Nivolumab, Pembrolizumab, or Cemiplimab. In some aspects, a PD-L1 inhibitor can be, but is not limited to, Atezolizumab, Avelumab, or Durvalumab. In some aspects, a CTLA-4 inhibitor can be, but is not limited to, ipilimumab or tremelimumab.

### D. Chemotherapies

The methods disclosed herein can also comprise administering one or more chemotherapies to a subject. In some aspects, the chemotherapy can be an alkylating agent, plant alkaloid, antimetabolite, anthracycline, topoisomerase inhibitor or corticosteroid.

In some aspects, the chemotherapy can be one or more of Altretamine, Bendamustine, Busulfan, Carboplatin, Chlorambucil, Cisplatin, Cyclophosphamide, Dacarbazine, Ifosfamide, Mechlorethamine, Melphalan, Oxaliplatin, Procarbazine, Temozolomide, Thiotepa, Trabectedin, Carmustine, Lomustine, Streptozocin, 5-fluorouracil, 6-mercaptopurine, Azacitidine, Capecitabine, Cladribine, Clofarabine, Cytarabine, Decitabine, Floxuridine, Fludarabine, Gemcitabine, Hydroxyurea, Methotrexate, Nelarabine, Pemetrexed, Pentostatin, Pralatrexate, Thioguanine, Trifluridine/tipiracil combination, Etoposide, Irinotecan, Irinotecan liposomal, Mitoxantrone, Teniposide, Topotecan, Cabazitaxel, Docetaxel, Nab-paclitaxel, Paclitaxel, Vinblastine, Vincristine, Vincristine liposomal, Vinorelbine, Daunorubicin, Doxorubicin, Doxorubicin liposomal, Epirubicin, Idarubicin, Mitoxantrone, Valrubicin, Bleomycin, Dactinomycin, Mitomycin-C, all-trans-retinoic acid, Arsenic trioxide, Asparaginase, Eribulin, Ixabepilone, Mitotane, Omacetaxine, Pegaspargase, Procarbazine, Romidepsin, Vorinostat, Dexamethasone, Hydrocortisone, Methylprednisolone, Prednisolone, or Prednisone.

### E. Methods

### 1. Methods of Increasing Survival

i. Alternating Electric Field Plus Checkpoint Inhibitor

Disclosed are methods of increasing survival of a subject having cancer comprising applying an alternating electric field to a target site of the subject for a period of time, the alternating electric field having a frequency and field strength, wherein the target site comprises one or more cancer cells, and administering a therapeutically effective amount of a checkpoint inhibitor to the subject. In some aspects, the increased survival is increased progression free survival.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject. In some aspects, the disclosed methods of increasing survival of a subject having non-small cell lung cancer further comprise screening the subject to determine whether the administering the therapeutically effective amount of a checkpoint inhibitor is effective in increasing survival or treating the subject. In some aspects, the disclosed methods of increasing survival of a subject having non-small cell lung cancer further comprise administering a chemotherapy to the subject. In some aspects, the chemotherapy can be an alkylating agent, plant alkaloid, antimetabolite, anthracycline, topoisomerase inhibitor or corticosteroid. In some aspects, the chemotherapy can be one or more of Altretamine, Bendamustine, Busulfan, Carboplatin, Chlorambucil, Cisplatin, Cyclophosphamide, Dacarbazine, Ifosfamide, Mechlorethamine, Melphalan, Oxaliplatin, Procarbazine, Temozolomide, Thiotepa, Trabectedin, Carmustine, Lomustine, Streptozocin, 5-fluorouracil, 6-mercaptopurine, Azacitidine, Capecitabine, Cladribine, Clofarabine, Cytarabine, Decitabine, Floxuridine, Fludarabine, Gemcitabine, Hydroxyurea, Methotrexate, Nelarabine, Pemetrexed, Pentostatin, Pralatrexate, Thioguanine, Trifluridine/tipiracil combination, Etoposide, Irinotecan, Irinotecan liposomal, Mitoxantrone, Teniposide, Topotecan, Cabazitaxel, Docetaxel, Nab-paclitaxel, Paclitaxel, Vinblastine, Vincristine, Vincristine liposomal, Vinorelbine, Daunorubicin, Doxorubicin, Doxorubicin liposomal, Epirubicin, Idarubicin, Mitoxantrone, Valrubicin, Bleomycin, Dactinomycin, Mitomycin-C, all-trans-retinoic acid, Arsenic trioxide, Asparaginase, Eribulin, Ixabepilone, Mitotane, Omacetaxine, Pegaspargase, Procarbazine, Romidepsin, Vorinostat, Dexamethasone, Hydrocortisone, Methylprednisolone, Prednisolone, or Prednisone.

In some aspects, the disclosed methods of increasing survival of a subject having non-small cell lung cancer increase survival by 4, 5, 6, 7, 8, 9 or more months when compared to a subject not treated according to the disclosed methods.

In some aspects, the checkpoint inhibitor can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, Tremelimumab, Sintilimab (formerly IB1308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In some aspects, the checkpoint inhibitor is a PD-1 inhibitor or PDL-1 inhibitor. In some aspects, a PD-1 inhibitor can be, but is not limited to, Nivolumab, Pembrolizumab, or Cemiplimab. In some aspects, a PD-L1 inhibitor can be, but is not limited to, Atezolizumab, Avelumab, or Durvalumab. In some aspects, nivolumab can be administered intravenously at a dose of 240 mg every 2 weeks or 480 mg every 4 weeks, or as a weight-based dose. In some aspects, pembrolizumab can be administered in a dose of 200 mg every 3 weeks, or 400 mg every 6 weeks as an intravenous infusion over 30 minutes, or as a weight-based dose. In some aspects, atezolizumab can be administered at 840 mg every 2 weeks, 1200 mg every 3 weeks, or 1680 mg every 4 weeks, as an intravenous infusion over 1 hour.

In some aspects, the cancer is metastatic non-small cell lung cancer (NSCLC). In some aspects, the NSCLC is a squamous cell NSCLC or a non-squamous cell NSCLC.

In some aspects, the increased survival is compared to survival of a subject administered the same therapeutically effective amount checkpoint inhibitor but without applying alternating electric field.

In some aspects, the increased survival of the subject is increased progression free survival. Thus, in some aspects, the increased progression free survival is compared to progression free survival of a subject administered the same therapeutically effective amount checkpoint inhibitor but without applying alternating electric field.

In some aspects, the increased progression free survival is an increased progression free survival of at least 2, 3, 4, 5, 6, 7, 8, 9, 10, 11, or 12 months. In some aspects, the increased progression free survival is an increased progression free survival of at least 4 months. In some aspects, the increased progression free survival is based on a median progression free survival of at least 2, 3, 4, 5, 6, 7, 8, 9, 10, 11, or 12 months. Thus, in some aspects, every patient may not have at least 2, 3, 4, 5, 6, 7, 8, 9, 10, 11, or 12 months of progression free survival but the median progression free survival is at least 2, 3, 4, 5, 6, 7, 8, 9, 10, 11, or 12 months.

In some aspects, the increased survival in a subject having squamous cell NSCLC is an increased survival of at least 3 months compared to a subject having squamous cell NSCLC administered the same therapeutically effective amount checkpoint inhibitor but without applying alternating electric field. In some aspects, a subject having squamous cell NSCLC can survive 9 to 18 months after treatment with alternating electric field and checkpoint inhibitor.

In some aspects, the increased survival in subject having non-squamous cell NSCLC is an increased survival of at least 2 months compared to a subject having non-squamous cell NSCLC administered the same therapeutically effective amount checkpoint inhibitor but without applying alternating electric field. In some aspects, a subject having non-squamous cell NSCLC can survive 8 to 20 months after treatment with alternating electric field and checkpoint inhibitor.

Also disclosed are methods of increasing survival of a subject having cancer comprising applying an alternating electric field to a target site of the subject for a period of time, the alternating electric field having a frequency and field strength, wherein the target site comprises one or more cancer cells; administering a therapeutically effective amount of a checkpoint inhibitor to the subject; and further comprising administering a taxane. In some aspects, the taxane is docetaxel, paclitaxel, cabazitaxel, abraxane, or a combination thereof.

In some aspects, the alternating electric field is applied before, after, or simultaneously with administering the checkpoint inhibitor. In some aspects, the step of applying the alternating electric fields begins at least one hour before a checkpoint inhibitor is administered. In some aspects, when the checkpoint inhibitor is administered before applying the alternating electric fields, the cells are surprisingly sensitized to the alternating electric fields, thereby increasing the efficacy of the alternating electric fields. In some aspects, the step of applying the alternating electric fields begins at least 30 minutes before administering a checkpoint inhibitor. In some aspects, applying the alternating electric fields simultaneously can mean applying within 5, 10, 15, 20, 25, 30, 35, 40, 45, 50, 55, or 60 minutes before or after administering an checkpoint inhibitor. In some aspects, the alternating electric fields can be applied and the checkpoint inhibitor administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the checkpoint inhibitor is administered intratumorally, intracranially, intraventricularly, intrathecally, epidurally, intradurally, intravascularly, intravenously, intraarterially, intramuscularly, subcutaneously, intraperitoneally, orally, intranasally, topically, via intratumor injection, or via inhalation.

In some aspects, the frequency and/or field strength of the alternating electric field can be any of those described herein and can be applied in any of the manners described herein. In some aspects, the frequency of the alternating electric field is between 50 kHz and 1 MHz. In some aspects, the frequency of the alternating electric field is between 100 kHz and 1 MHz. In some aspects, the frequency of the alternating electric field is about 150 or 250 kHz. In some aspects, the alternating electric field has a field strength of between 0.5 and 10 V/cm RMS. In some aspects, the alternating electric field has a field strength of about 0.9 V/cm RMS.

In some aspects, the disclosed methods of increasing survival of a subject having non-small cell lung cancer comprise applying an alternating electric field occurs 1, 2, 3, 4, 5, 6, or 7 days prior to administering the checkpoint inhibitor. In some aspects, the disclosed methods of increasing survival of a subject having non-small cell lung cancer comprise applying an alternating electric field occurs 1, 2, 3, 4, 5, 6, or 7 days after administering the checkpoint inhibitor. In some aspects, applying alternating electric fields occurs 1, 2, 3, or 4 weeks prior to administering the checkpoint inhibitor. In some aspects, applying alternating electric fields occurs 1, 2, 3, or 4 weeks after administering the checkpoint inhibitor. In some aspects, the alternating electric fields and the checkpoint inhibitor are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the checkpoint inhibitor is present in the bloodstream prior to applying the alternating electric field.

In some aspects, the disclosed methods of increasing survival of a subject having non-small cell lung cancer, further comprise discontinuing the alternating electrical field during the method. In some aspects, the disclosed methods of increasing survival of a subject having non-small cell lung cancer, the alternating electric field can be applied discontinuously over the course of treatment. For example, the alternating electrical field can be applied less than 24 hours a day and 7 days a week. In some aspect, the alternating electrical field can be applied at least 6, 7, 8, 9, 10, 11, 12, 13, 14, 15, 16, 17, 18 or 19 hours a day or more.

In some aspects, administering a checkpoint inhibitor is performed 1, 2, 3, 4, 5, 6, 7, 8, 9, or 10 days after applying an alternating electric field is performed. In some aspects, administering a checkpoint inhibitor is performed simultaneously with applying an alternating electric field. In some aspects, administering a checkpoint inhibitor is performed within hours, days, or weeks of applying an alternating electric field.

In some aspects, the alternating electric field can be induced by an applied voltage of at least 50 V RMS. In some aspects, the alternating electric field can be induced by an applied voltage of at least 25-200 V RMS. In some aspects, the alternating electric field can be induced by an applied voltage of at least about 5, 10, 15, 20, 25, 50, 75, 100, 125, 150, 175, 200, 225, 250, 275 or 300 V RMS.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

Disclosed herein is the use of an alternating electric field and a checkpoint inhibitor to increase survival of a subject having non-small cell lung cancer.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda). In some aspects, the disclosed methods of increasing survival of a subject having non-small cell lung cancer further comprise screening the subject to determine whether the administering the therapeutically effective amount of a checkpoint inhibitor is effective in increasing survival or treating the subject. In some aspects, the disclosed methods of increasing survival of a subject having non-small cell lung cancer further comprise administering a chemotherapy to the subject. In some aspects, the chemotherapy can be administered prior to, after, or simultaneously with the alternating electric field. In some aspects, the chemotherapy can be administered prior to, after, or simultaneously with the checkpoint inhibitor.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda). In some aspects, the subject has been diagnosed with radiological progression of the non-small cell lung cancer on or after the previous treatment with the checkpoint inhibitor. In some aspects, the subject progressed to metastatic disease within 6 months of completing the previous treatment with the checkpoint inhibitor.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, and wherein the subject was not previously treated with a non-platinum-based chemotherapy or a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, and wherein the subject was not previously treated with a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, and wherein the subject has not had a cerebrovascular accident within six (6) months prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, and wherein the subject has not had a non-NSCLC malignancy within 3 years prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, further comprising screening the subject to determine whether the administering the therapeutically effective amount of a checkpoint inhibitor is effective in increasing survival of the subject. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda). In some aspects, the disclosed methods of increasing survival of a subject having non-small cell lung cancer further comprise screening the subject to determine whether the administering the therapeutically effective amount of a checkpoint inhibitor is effective in increasing survival or treating the subject. In some aspects, the disclosed methods of increasing survival of a subject having non-small cell lung cancer further comprise administering a chemotherapy to the subject.

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and the chemotherapy. In some aspects, the subject has been diagnosed with radiological progression of the non-small cell lung cancer on or after the previous treatment with the checkpoint inhibitor and chemotherapy. In some aspects, the subject progressed to metastatic disease within 6 months of completing the previous treatment with the checkpoint inhibitor and chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and the chemotherapy, wherein the subject was not previously treated with a non-platinum-based chemotherapy or a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and chemotherapy, wherein the subject has not had a cerebrovascular accident within six (6) months prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and chemotherapy, wherein the subject has not had a non-NSCLC malignancy within 3 years prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and chemotherapy, further comprising screening the subject to determine whether the administering the therapeutically effective amount of a checkpoint inhibitor is effective in increasing survival of the subject. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of increasing survival of a subject having cancer comprising applying an alternating electric field to a target site of the subject for a period of time, the alternating electric field having a frequency and field strength, wherein the target site comprises one or more cancer cells, and administering a therapeutically effective amount of a checkpoint inhibitor to the subject, wherein the cancer is metastatic non-small cell lung cancer, wherein the subject was previously treated for non-small cell lung cancer with systemic therapy including treatment with a checkpoint inhibitor and platinum-based-chemotherapy, and wherein the non-small cell lung cancer in the subject progressed on or after the previous systemic treatment, wherein the frequency of the alternating electric field is 150 kHz. In some aspects, the checkpoint inhibitor administered in the step of administering the therapeutically effective amount of the checkpoint inhibitor to the subject is a PD-1 inhibitor, PD-L1 inhibitor, or CTLA-4 inhibitor. In some aspects, a PD-1 inhibitor can be, but is not limited to, Nivolumab, Pembrolizumab, or Cemiplimab. In some aspects, a PD-L1 inhibitor can be, but is not limited to, Atezolizumab, Avelumab, or Durvalumab. In some aspects, a CTLA-4 inhibitor can be, but is not limited to, ipilimumab or tremelimumab. In some aspects, the checkpoint inhibitor administered in the step of administering the therapeutically effective amount of the checkpoint inhibitor to the subject is Pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed herein is the use of an alternating electric field and a checkpoint inhibitor to increase survival of a subject having non-small cell lung cancer. In some aspects, disclosed herein is the use of an alternating electric field and a checkpoint inhibitor in combination with a chemotherapy to increase survival of a subject having non-small cell lung cancer. In some aspects, disclosed herein is the use of an alternating electric field and a checkpoint inhibitor to increase survival of a subject having non-small cell lung cancer, wherein the subject was previously treated with a checkpoint inhibitor, wherein the non-small cell lung cancer in the subject progressed on or after the previous treatment with the checkpoint inhibitor. In some aspects, the subject has been diagnosed with radiological progression of the non-small cell lung cancer on or after the previous treatment with the checkpoint inhibitor. In some aspects, the subject progressed to metastatic disease within 6 months of completing the previous treatment with the checkpoint inhibitor. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to increase survival of a subject having non-small cell lung cancer, wherein the subject was previously treated with a checkpoint inhibitor, wherein the non-small cell lung cancer in the subject progressed on or after the previous treatment with the checkpoint inhibitor, wherein the subject was not previously treated with a non-platinum-based chemotherapy or a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to increase survival of a subject having non-small cell lung cancer, wherein the subject was previously treated with a checkpoint inhibitor, wherein the non-small cell lung cancer in the subject progressed on or after the previous treatment with the checkpoint inhibitor, wherein the subject was not previously treated with a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to increase survival of a subject having non-small cell lung cancer, wherein the subject was previously treated with a checkpoint inhibitor, wherein the non-small cell lung cancer in the subject progressed on or after the previous treatment with the checkpoint inhibitor, wherein the subject has not had a cerebrovascular accident within six (6) months prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to increase survival of a subject having non-small cell lung cancer, wherein the subject was previously treated with a checkpoint inhibitor, wherein the non-small cell lung cancer in the subject progressed on or after the previous treatment with the checkpoint inhibitor, wherein the subject has not had a non-NSCLC malignancy within 3 years prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed herein is the use of an alternating electric field and a checkpoint inhibitor to increase survival of a subject having non-small cell lung cancer. In some aspects, disclosed herein is the use of an alternating electric field and a checkpoint inhibitor in combination with a chemotherapy to increase survival of a subject having non-small cell lung cancer. In some aspects, disclosed herein is the use of an alternating electric field and a checkpoint inhibitor to increase survival of a subject having non-small cell lung cancer, wherein the subject was previously treated with a checkpoint inhibitor and chemotherapy, wherein the non-small cell lung cancer in the subject progressed on or after the previous treatment with the checkpoint inhibitor and chemotherapy. In some aspects, the subject has been diagnosed with radiological progression of the non-small cell lung cancer on or after the previous treatment with the checkpoint inhibitor and chemotherapy. In some aspects, the subject progressed to metastatic disease within 6 months of completing the previous treatment with the checkpoint inhibitor and chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to increase survival of a subject having non-small cell lung cancer, wherein the subject was previously treated with a checkpoint inhibitor and chemotherapy, wherein the non-small cell lung cancer in the subject progressed on or after the previous treatment with the checkpoint inhibitor and chemotherapy, wherein the subject was not previously treated with a non-platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to increase survival of a subject having non-small cell lung cancer, wherein the subject was previously treated with a checkpoint inhibitor, wherein the non-small cell lung cancer in the subject progressed on or after the previous treatment with the checkpoint inhibitor and chemotherapy, wherein the subject was not previously treated with a platinum-based chemotherapy.

In some aspects, disclosed herein is the use of an alternating electric field and a checkpoint inhibitor to increase survival of a subject having non-small cell lung cancer, wherein the subject was previously treated with a checkpoint inhibitor and chemotherapy, wherein the non-small cell lung cancer in the subject progressed on or after the previous treatment with the checkpoint inhibitor and chemotherapy, wherein the subject has not had a cerebrovascular accident within six (6) months prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to increase survival of a subject having non-small cell lung cancer, wherein the subject was previously treated with a checkpoint inhibitor and chemotherapy, wherein the non-small cell lung cancer in the subject progressed on or after the previous treatment with the checkpoint inhibitor and chemotherapy, wherein the subject has not had a non-NSCLC malignancy within 3 years prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

ii. Alternating Electric Field Plus Taxane

Disclosed are methods of increasing survival of a subject having cancer comprising applying an alternating electric field to a target site of the subject for a period of time, the alternating electric field having a frequency and field strength, wherein the target site comprises one or more cancer cells, and administering a therapeutically effective amount of a taxane to the subject.

In some aspects, the taxane is docetaxel, paclitaxel, cabazitaxel, abraxane, or a combination thereof. In some aspects, docetaxel can be administered intravenously as a single agent at 75 mg/m2 over 1 hour every 3 weeks. In some aspects, paclitaxel can be administered intravenously as a single agent at 175 mg/m2 over 3 hour every 3 weeks. In some aspects, cabazitaxel can be administered intravenously as a single agent at 20 mg/m2 over 1 hour every 3 weeks. In some aspects, abraxane can be administered intravenously as a single agent at 260 mg/m2 over 30 minutes every 3 weeks.

In some aspects, the cancer is metastatic non-small cell lung cancer (NSCLC). In some aspects, the NSCLC is a squamous cell NSCLC or a non-squamous cell NSCLC.

In some aspects, the increased survival is compared to survival of a subject administered the same therapeutically effective amount of taxane but without applying alternating electric field.

In some aspects, the increased survival of the subject is increased progression free survival. Thus, in some aspects, the increased progression free survival is compared to progression free survival of a subject administered the same therapeutically effective amount of taxane but without applying alternating electric field.

In some aspects, the increased progression free survival is an increased progression free survival of at least 2, 3, 4, 5, 6, 7, 8, 9, 10, 11, or 12 months. In some aspects, the increased progression free survival is an increased progression free survival of at least 4 months. In some aspects, the increased progression free survival is based on a median progression free survival of at least 2, 3, 4, 5, 6, 7, 8, 9, 10, 11, or 12 months. Thus, in some aspects, every patient may not have at least 2, 3, 4, 5, 6, 7, 8, 9, 10, 11, or 12 months of progression free survival but the median progression free survival is at least 2, 3, 4, 5, 6, 7, 8, 9, 10, 11, or 12 months.

In some aspects, the increased survival in a subject having squamous cell NSCLC is an increased survival of at least 3 months compared to a subject having squamous cell NSCLC administered the same therapeutically effective amount of taxane but without applying alternating electric field. In some aspects, a subject having squamous cell NSCLC can survive 9 to 18 months after treatment with alternating electric field and a taxane.

In some aspects, the increased survival in subject having non-squamous cell NSCLC is an increased survival of at least 2 months compared to a subject having non-squamous cell NSCLC administered the same therapeutically effective amount of taxane but without applying alternating electric field. In some aspects, a subject having non-squamous cell NSCLC can survive 8 to 20 months after treatment with alternating electric field and taxane. Disclosed are methods of increasing survival of a subject having cancer comprising applying an alternating electric field to a target site of the subject for a period of time, the alternating electric field having a frequency and field strength, wherein the target site comprises one or more cancer cells; administering a therapeutically effective amount of a taxane to the subject; and further comprising administering a therapeutically effective amount of a checkpoint inhibitor to the subject. In some aspects, the checkpoint inhibitor is a PD-1 inhibitor or PDL-1 inhibitor. In some aspects, a PD-1 inhibitor can be, but is not limited to, Nivolumab, Pembrolizumab, or Cemiplimab. In some aspects, a PD-1 inhibitor can be, but is not limited to, Atezolizumab, Avelumab, or Durvalumab.

### 2. Methods of Treating a Subject

i. Alternating Electric Field Plus Checkpoint Inhibitor

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a checkpoint inhibitor to the subject.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject. In some aspects, the disclosed methods of treating a subject having non-small cell lung cancer further comprise screening the subject to determine whether the administering the therapeutically effective amount of a checkpoint inhibitor is effective in increasing survival or treating the subject. In some aspects, the disclosed methods of treating a subject having non-small cell lung cancer further comprise administering a chemotherapy to the subject.

In some aspects, the checkpoint inhibitor can be, but is not limited to, ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio), or a combination thereof. In some aspects, the checkpoint inhibitor can be, but is not limited to, Tremelimumab, Sintilimab (formerly IBI308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189, or a combination thereof.

In some aspects, applying an alternating electric field occurs 1, 2, 3, 4, 5, 6, or 7 days prior to administering the checkpoint inhibitor. In some aspects, applying an alternating electric field occurs 1, 2, 3, 4, 5, 6, or 7 days after administering the checkpoint inhibitor. In some aspects, applying alternating electric fields occurs 1, 2, 3, or 4 weeks prior to administering the checkpoint inhibitor. In some aspects, applying alternating electric fields occurs 1, 2, 3, or 4 weeks after administering the checkpoint inhibitor. In some aspects, the alternating electric fields and the checkpoint inhibitor are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the checkpoint inhibitor is present in the bloodstream prior to applying the alternating electric field.

In some aspects, the alternating electric field can have a frequency and field strength. In some aspects, the frequency of the alternating electric field is between 50 and 1 MHz. In some aspects, the frequency of the alternating electric field is 100-1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field may be induced by an applied voltage of at least 50 V RMS. In some aspects, the alternating electric field can be induced by an applied voltage of at least 25-200 V RMS. In some aspects, the alternating electric field can be induced by an applied voltage of at least about 5, 10, 15, 20, 25, 50, 75, 100, 125, 150, 175, 200, 225, 250, 275 or 300 V RMS.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

In some aspects, the disclosed methods of treating a subject having non-small cell lung cancer, further comprise discontinuing the alternating electrical field during the method. In some aspects, the disclosed methods of treating a subject having non-small cell lung cancer, the alternating electric field can be applied discontinuously over the course of treatment. For example, the alternating electrical field can be applied less than 24 hours a day and 7 days a week. In some aspect, the alternating electrical field can be applied at least 6, 7, 8, 9, 10, 11, 12, 13, 14, 15, 16, 17, 18 or 19 hours a day or more.

Disclosed herein is the use of an alternating electric field and a checkpoint inhibitor to treat a subject having non-small cell lung cancer.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor. In some aspects, the checkpoint inhibitor the subject was previously treated with is the same checkpoint inhibitor administered in combination with the alternating electric field. In some aspects, the checkpoint inhibitor the subject was previously treated with is pembrolizumab (Keytruda) and the checkpoint inhibitor administered in combination with the alternating electric field is pembrolizumab (Keytruda). In some aspects, the disclosed methods of treating a subject having non-small cell lung cancer further comprise screening the subject to determine whether the administering the therapeutically effective amount of a checkpoint inhibitor is effective in increasing survival or treating the subject. In some aspects, the disclosed methods of treating a subject having non-small cell lung cancer further comprise administering a chemotherapy to the subject. In some aspects, the chemotherapy can be administered prior to, after, or simultaneously with the alternating electric field. In some aspects, the chemotherapy can be administered prior to, after, or simultaneously with the checkpoint inhibitor.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor. In some aspects, the subject has been diagnosed with radiological progression of the non-small cell lung cancer on or after the previous treatment with the checkpoint inhibitor. In some aspects, the subject progressed to metastatic disease within 6 months of completing the previous treatment with the checkpoint inhibitor. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, wherein the subject was not previously treated with a non-platinum-based chemotherapy or a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, wherein the subject was not previously treated with a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, wherein the subject has not had a cerebrovascular accident within six (6) months prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, wherein the subject has not had a non-NSCLC malignancy within 3 years prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor, wherein the non-small cell lung cancer in the subject progressed on or after the previous treatment with the checkpoint inhibitor, further comprising screening the subject to determine whether the administering the therapeutically effective amount of a checkpoint inhibitor is effective in treating the subject. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda). In some aspects, the disclosed methods of treating a subject having non-small cell lung cancer further comprise screening the subject to determine whether the administering the therapeutically effective amount of a checkpoint inhibitor is effective in increasing survival or treating the subject. In some aspects, the disclosed methods of increasing treating a subject having non-small cell lung cancer further comprise administering a chemotherapy to the subject.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and the chemotherapy. In some aspects, the subject has been diagnosed with radiological progression of the non-small cell lung cancer on or after the previous treatment with the checkpoint inhibitor and chemotherapy. In some aspects, the subject progressed to metastatic disease within 6 months of completing the previous treatment with the checkpoint inhibitor and chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and the chemotherapy, wherein the subject was not previously treated with a non-platinum-based chemotherapy or a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and chemotherapy, wherein the subject has not had a cerebrovascular accident within six (6) months prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and chemotherapy, wherein the subject has not had a non-NSCLC malignancy within 3 years prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a checkpoint inhibitor to the subject, wherein the subject was previously treated with a checkpoint inhibitor and chemotherapy, wherein the non-small cell lung cancer in the subject progressed on or after the previous treatment with the checkpoint inhibitor and chemotherapy, further comprising screening the subject to determine whether the administering the therapeutically effective amount of a checkpoint inhibitor is effective in treating the subject. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a checkpoint inhibitor to the subject, wherein the cancer is metastatic non-small cell lung cancer, wherein the subject was previously treated for non-small cell lung cancer with systemic therapy including treatment with a checkpoint inhibitor and platinum-based-chemotherapy, and wherein the non-small cell lung cancer in the subject progressed on or after the previous systemic treatment, wherein the frequency of the alternating electric field is 150 kHz. In some aspects, the checkpoint inhibitor administered in the step of administering the therapeutically effective amount of the checkpoint inhibitor to the subject is a PD-1 inhibitor, PD-L1 inhibitor, or CTLA-4 inhibitor. In some aspects, a PD-1 inhibitor can be, but is not limited to, Nivolumab, Pembrolizumab, or Cemiplimab. In some aspects, a PD-L1 inhibitor can be, but is not limited to, Atezolizumab, Avelumab, or Durvalumab. In some aspects, a CTLA-4 inhibitor can be, but is not limited to, ipilimumab or tremelimumab. In some aspects, the checkpoint inhibitor administered in the step of administering the therapeutically effective amount of the checkpoint inhibitor to the subject is Pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed herein is the use of an alternating electric field and a checkpoint inhibitor to treating a subject having non-small cell lung cancer. In some aspects, disclosed herein is the use of an alternating electric field and a checkpoint inhibitor in combination with a chemotherapy to treat a subject having non-small cell lung cancer. In some aspects, disclosed herein is the use of an alternating electric field and a checkpoint inhibitor to treat a subject having non-small cell lung cancer, wherein the subject was previously treated with a checkpoint inhibitor, wherein the non-small cell lung cancer in the subject progressed on or after the previous treatment with the checkpoint inhibitor. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda). In some aspects, the subject has been diagnosed with radiological progression of the non-small cell lung cancer on or after the previous treatment with the checkpoint inhibitor. In some aspects, the subject progressed to metastatic disease within 6 months of completing the previous treatment with the checkpoint inhibitor.

In some aspects, disclosed herein is the use of an alternating electric field and a checkpoint inhibitor to treat a subject having non-small cell lung cancer, wherein the subject was previously treated with a checkpoint inhibitor, wherein the non-small cell lung cancer in the subject progressed on or after the previous treatment with the checkpoint inhibitor, wherein the subject was not previously treated with a non-platinum-based chemotherapy or a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to treat a subject having non-small cell lung cancer, wherein the subject was previously treated with a checkpoint inhibitor, wherein the non-small cell lung cancer in the subject progressed on or after the previous treatment with the checkpoint inhibitor, wherein the subject was not previously treated with a platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to treat a subject having non-small cell lung cancer, wherein the subject was previously treated with a checkpoint inhibitor, wherein the non-small cell lung cancer in the subject progressed on or after the previous treatment with the checkpoint inhibitor, wherein the subject has not had a cerebrovascular accident within six (6) months prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to treat a subject having non-small cell lung cancer, wherein the subject was previously treated with a checkpoint inhibitor, wherein the non-small cell lung cancer in the subject progressed on or after the previous treatment with the checkpoint inhibitor, wherein the subject has not had a non-NSCLC malignancy within 3 years prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

Disclosed herein is the use of an alternating electric field and a checkpoint inhibitor to treat a subject having non-small cell lung cancer. In some aspects, disclosed herein is the use of an alternating electric field and a checkpoint inhibitor in combination with a chemotherapy to treat a subject having non-small cell lung cancer. In some aspects, disclosed herein is the use of an alternating electric field and a checkpoint inhibitor to treat a subject having non-small cell lung cancer, wherein the subject was previously treated with a checkpoint inhibitor and chemotherapy, wherein the non-small cell lung cancer in the subject progressed on or after the previous treatment with the checkpoint inhibitor and chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda). In some aspects, the subject has been diagnosed with radiological progression of the non-small cell lung cancer on or after the previous treatment with the checkpoint inhibitor and chemotherapy. In some aspects, the subject progressed to metastatic disease within 6 months of completing the previous treatment with the checkpoint inhibitor and chemotherapy.

In some aspects, disclosed herein is the use of an alternating electric field and a checkpoint inhibitor to treat a subject having non-small cell lung cancer, wherein the subject was previously treated with a checkpoint inhibitor and chemotherapy, wherein the non-small cell lung cancer in the subject progressed on or after the previous treatment with the checkpoint inhibitor and chemotherapy, wherein the subject was not previously treated with a non-platinum-based chemotherapy. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint inhibitor to treat a subject having non-small cell lung cancer, wherein the subject was previously treated with a checkpoint inhibitor, wherein the non-small cell lung cancer in the subject progressed on or after the previous treatment with the checkpoint inhibitor and chemotherapy, wherein the subject was not previously treated with a platinum-based chemotherapy.

In some aspects, disclosed herein is the use of an alternating electric field and a checkpoint inhibitor to treat a subject having non-small cell lung cancer, wherein the subject was previously treated with a checkpoint inhibitor and chemotherapy, wherein the non-small cell lung cancer in the subject progressed on or after the previous treatment with the checkpoint inhibitor and chemotherapy, wherein the subject has not had a cerebrovascular accident within six (6) months prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

In some aspects, disclosed herein is the use of an alternating electric field and a checkpoint treat a subject a subject having non-small cell lung cancer, wherein the subject was previously treated with a checkpoint inhibitor and chemotherapy, wherein the non-small cell lung cancer in the subject progressed on or after the previous treatment with the checkpoint inhibitor and chemotherapy, wherein the subject has not had a non-NSCLC malignancy within 3 years prior to administration of the alternating electric field. In some aspects, the checkpoint inhibitor the subject was previously treated with is the same checkpoint inhibitor administered in combination with the alternating electric field. In some aspects, checkpoint inhibitor the subject was previously treated with is pembrolizumab (Keytruda) and the checkpoint inhibitor administered in combination with the alternating electric field is pembrolizumab (Keytruda). In some aspects, checkpoint inhibitor the subject was previously treated with is pembrolizumab (Keytruda) and the checkpoint inhibitor administered in combination with the alternating electric field is pembrolizumab (Keytruda) and the subject progressed on or after the previous treatment with pembrolizumab (Keytruda).

ii. Alternating Electric Field Plus Taxane

Disclosed are methods of treating a subject having cancer comprising applying an alternating electric field to a target site of the subject for a period of time, the alternating electric field having a frequency and field strength, wherein the target site comprises one or more cancer cells, and administering a therapeutically effective amount of a taxane to the subject.

Disclosed are methods of treating a subject having non-small cell lung cancer comprising: applying an alternating electric field to a target site of the subject for a period of time, the alternating electric field having a frequency and field strength, wherein the target site comprises one or more non-small cell lung cancer cells, and administering a therapeutically effective amount of a taxane to the subject. In some aspects, the disclosed methods of treating a subject having non-small cell lung cancer further comprise screening the subject to determine whether the administering the therapeutically effective amount of a taxane is effective in increasing survival or treating the subject. In some aspects, the disclosed methods of treating a subject having non-small cell lung cancer further comprise administering a chemotherapy to the subject.

In some aspects, the taxane is docetaxel, paclitaxel, cabazitaxel, abraxane, or a combination thereof. In some aspects, docetaxel can be administered intravenously as a single agent at 75 mg/m2 over 1 hour every 3 weeks. In some aspects, paclitaxel can be administered intravenously as a single agent at 175 mg/m2 over 3 hour every 3 weeks. In some aspects, cabazitaxel can be administered intravenously as a single agent at 20 mg/m2 over 1 hour every 3 weeks. In some aspects, abraxane can be administered intravenously as a single agent at 260 mg/m2 over 30 minutes every 3 weeks.

In some aspects, the cancer is metastatic non-small cell lung cancer (NSCLC). In some aspects, the NSCLC is a squamous cell NSCLC or a non-squamous cell NSCLC.

In some aspects, applying an alternating electric field occurs 1, 2, 3, 4, 5, 6, or 7 days prior to administering the taxane. In some aspects, applying an alternating electric field occurs 1, 2, 3, 4, 5, 6, or 7 days after administering the taxane. In some aspects, applying alternating electric fields occurs 1, 2, 3, or 4 weeks prior to administering the taxane. In some aspects, applying alternating electric fields occurs 1, 2, 3, or 4 weeks after administering the taxane. In some aspects, the alternating electric fields and the taxane are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the taxane is present in the bloodstream prior to applying the alternating electric field.

In some aspects, the alternating electric field can have a frequency and field strength. In some aspects, the frequency of the alternating electric field is between 50 and 1 MHz. In some aspects, the frequency of the alternating electric field is 100-1 MHz. In some aspects, the frequency of the alternating electric field is 100-500 kHz. In some aspects, the frequency of the alternating electric field is 200 kHz. In some aspects, the alternating electric field can be any of the ranges described herein.

The alternating electric field may be induced by an applied voltage of at least 50 V RMS. In some aspects, the alternating electric field can be induced by an applied voltage of at least 25-200 V RMS. In some aspects, the alternating electric field can be induced by an applied voltage of at least about 5, 10, 15, 20, 25, 50, 75, 100, 125, 150, 175, 200, 225, 250, 275 or 300 V RMS.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

In some aspects, the disclosed methods of treating a subject having non-small cell lung cancer, further comprise discontinuing the alternating electrical field during the method. In some aspects, the disclosed methods of treating a subject having non-small cell lung cancer, the alternating electric field can be applied discontinuously over the course of treatment. For example, the alternating electrical field can be applied less than 24 hours a day and 7 days a week. In some aspect, the alternating electrical field can be applied at least 6, 7, 8, 9, 10, 11, 12, 13, 14, 15, 16, 17, 18 or 19 hours a day or more.

Disclosed herein is the use of an alternating electric field and a taxane to treat a subject having non-small cell lung cancer.

### F. Subject Population

In some aspects of the disclosed methods, the subject population can be one or more of those described herein.

In some aspects of the disclosed methods, the subject has been diagnosed with radiological progression while on or after platinum-based systemic therapy. In some aspects, the platinum-based systemic therapy is nedaplatin, satraplatin, cisplatin, carboplatin, and oxaliplatin.

In some aspects of the disclosed methods, the subject progressed to metastatic disease within 6 months of completing a platinum-based systemic therapy.

In some aspects of the disclosed methods, the subject does not have a significant hematological, hepatic, renal, or cardiac dysfunction

In some aspects of the disclosed methods, the subject has not had a cerebrovascular accident within six (6) months prior to administration of the alternating electric field.

In some aspects of the disclosed methods, the subject has not had a non-NSCLC malignancy within 3 years prior to administration of the alternating electric field. In some aspects, the subject has not had a non-NSCLC other than stage I prostate cancer, non-melanoma skin cancer, or in situ cervical cancer or breast cancer malignancy within 3 years prior to administration of the alternating electric field.

In some aspects of the disclosed methods, the subject having cancer has a non-squamous disease. In some aspects, the subject has a non-squamous cancer or squamous cancer.

In some aspects of the disclosed methods, the subject having cancer has been previously treated with an immune checkpoint inhibitor.

In some aspects of the disclosed methods, the subject is a non-smoker, current smoker, or former smoker.

In some aspects of the disclosed methods, the subject has liver metastases.

In some aspects of the disclosed methods, the subject having cancer is at least 22 years of age.

In some aspects of the disclosed methods, the subject does not have a central nervous system metastases.

In some aspects of the disclosed methods, the subject does not have a clinically significant hematological, hepatic or renal dysfunction. In some aspects, clinically significant dysfunction refers to hematological, hepatic or renal dysfunction that does not alter (e.g., reduce) the quality of life or quality of function of the hematological, hepatic or renal system of the subject.

In some aspects of the disclosed methods, the subject does not have a second or third degree heart blockage, uncontrolled hypertension, congestive heart failure, a history of arrhythmia that is symptomatic or requires treatment, a history of pericarditis, or a history of interstitial lung disease.

In some aspects of the disclosed methods, the subject is not pregnant

### G. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits for treating cancer. In some aspects, the kit can comprise equipment for applying alternating electrical fields.

For example disclosed are kits comprising one or more of checkpoint inhibitors and one or more materials for delivering alternating electric fields, such as the Optune system. For example, disclosed are kits comprising one or more of ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio) and one or more materials for delivering alternating electric fields, such as the Optune system. For example, disclosed are kits comprising one or more of Tremelimumab, Sintilimab (formerly IBI308; Tyvyt), Tislelizumab (formerly BGB-A317), Toripalimab (formerly JS 001), Spartalizumab (formerly PRD001); Camrelizumab (formerly SHR1210), KN035, Cosibelimab (formerly CK-301), CA-170, or BMS-986189 and one or more materials for delivering alternating electric fields, such as the Optune system.

### EXAMPLES

### A. Example 1: Tumor Treating Fields in Pretreated Metastatic Non-Small Cell Lung Cancer

### 1. Methods

i. Study Design and Oversight

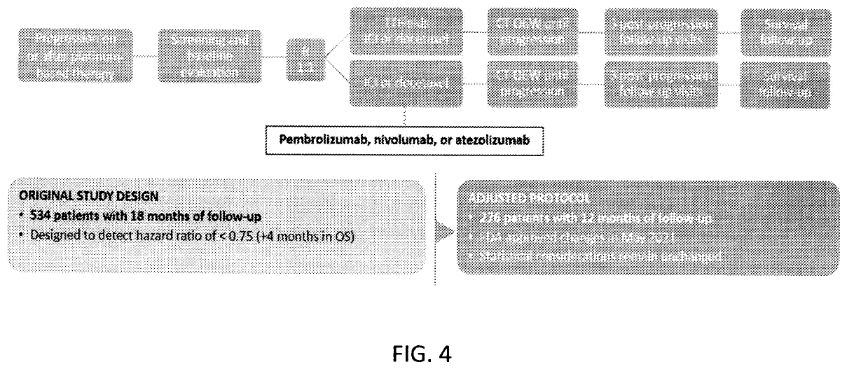

LUNAR was a prospective, phase 3, randomized, open-label, parallel study (EF-24; NCT02973789) of TTFields (150 kHz) therapy concomitant with standard-of-care systemic therapy (docetaxel or investigator's choice immune checkpoint inhibitor) in patients with metastatic NSCLC progressing on or after platinum therapy. LUNAR was conducted at 124 sites across 17 countries in North America, Europe, and Asia (Table Si) between February 2017 and November 2021. The study design is shown in FIG. 4, and the full study protocol is available online. A pilot study was also conducted demonstrating safety and feasibility of TTFields therapy concomitant with pemetrexed in patients with advanced NSCLC.¹⁸

The LUNAR study was designed by the sponsor (Novocure GmbH) and the investigators. Data were collected by the investigators and analyzed by sponsor-employed statisticians. The first draft of this manuscript was written by the authors with professional editorial support. All the authors contributed to data interpretation and vouch for completeness and accuracy, as well as fidelity of the study to the protocol.

An independent Data Monitoring Committee (DMC), comprised of an oncologist, pulmonologist, and statistician, monitored data, assessed results at an Interim Analysis, and provided recommendations to the sponsor based on the frequency of adverse events and duration of overall survival. The protocol and all amendments were approved by the relevant Ethics Committee and Competent Authority at each participating site. This study conformed to the ethical guidelines of the 1975 Declaration of Helsinki and was conducted in compliance with good clinical practice guidelines (EN ISO 14155:2011) and all appropriate national/regional regulations. All patients provided written informed consent.

### II. Patient Population

Eligible participants were adults (≥22 years of age) with a histological or cytological diagnosis of metastatic NSCLC that had shown radiological progression while on or after platinum-based systemic therapy (prior ICI permitted). Other than maintenance therapy, the most recent systemic therapy administered for advanced NSCLC was platinum-therapy. Patients who had progressed to metastatic disease within 6 months of completing platinum therapy in the adjuvant setting were also eligible. Patients had an Eastern Cooperative Oncology Group (ECOG) performance status (PS) of 0-2 and a life expectancy of more than 3 months. A protocol amendment allowed inclusion of neurologically stable patients with treated central nervous system metastases. Exclusion criteria included severe comorbidities (including clinically significant hematological, hepatic, renal, or cardiac dysfunction), a cerebrovascular accident within 6 months of randomization, or another (non-NSCLC) malignancy within 3 years of entering the study (excluding stage I prostate cancer, non-melanoma skin cancer, or in situ cervical cancer or breast cancer). Full eligibility criteria are in Table 4.

ii. procedures

Eligible patients were randomly assigned 1:1 (within 28 days of providing informed consent) to receive TTFields therapy to the thorax concomitant with investigator's choice of standard therapy (an immune checkpoint inhibitor [nivolumab, pembrolizumab, or atezolizumab] or docetaxel). Randomization was determined centrally using variable blocked randomization and an IxRS system, and stratified by tumor histology, treatment (docetaxel or an immune checkpoint inhibitor), and region (North America, Western Europe/Israel, and Eastern Europe).

Docetaxel was administered intravenously as a single agent at 75 mg/m2 over 1 hour every 3 weeks. Nivolumab was administered intravenously at a dose of 240 mg every 2 weeks or 480 mg every 4 weeks, or as a weight-based dose. Pembrolizumab was administered in a dose of 200 mg every 3 weeks, or 400 mg every 6 weeks as an intravenous infusion over 30 minutes, or as a weight-based dose. Atezolizumab was administered at 840 mg every 2 weeks, 1200 mg every 3 weeks, or 1680 mg every 4 weeks, as an intravenous infusion over 1 hour. All systemic therapies were administered until disease progression or unacceptable toxicity, dosing was adjusted per local standard of care.

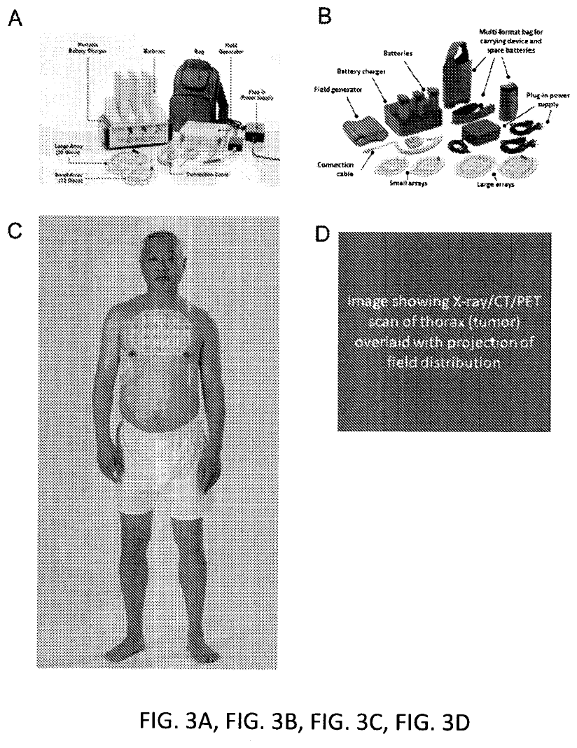

TTFields (150 kHz) were delivered continuously for an average of at least 18 hours per day with the NovoTTF device system (FIG. 3). Patients who initiated the study using the NovoTTF-100L system were offered the possibility to switch to the next generation NovoTTF-200T system, which is smaller and lighter. Array layouts were determined by the investigator and modified as needed throughout the treatment period, per computed tomography scans and provided clinical practice guidelines. Patients were trained by the investigator, other healthcare provider, or a Device Support Specialist (DSS; sponsor provided) to use the device. Arrays were replaced (and shifted by approximately 2 cm from the original position to minimize skin irritation) every 3-4 days. TTFields usage time (device-captured data) was reported monthly to investigators, presented as a percentage.

Follow-up visits were conducted every 6 weeks (+1 week) for radiological assessment of disease per Response Evaluation Criteria in Solid Tumors (RECIST) version 1.1 for patients receiving docetaxel, or immune-related (ir)RECIST for patients receiving an immune checkpoint inhibitor.

TTFields therapy was continued until progression in the thorax and/or liver or intolerable toxicity. Treatment breaks of up to 3 weeks were allowed for TTFields-related adverse events. Patients could continue to receive TTFields therapy, even if systemic therapy was discontinued due to progression outside of the thorax/liver or intolerable toxicity. Following progression, patients were offered investigator's choice of salvage therapy according to local practice.

Safety was assessed at each follow-up visit (from the time of randomization until 100 days after terminating study treatment), with adverse events reported according to National Cancer Institute Common Terminology Criteria for Adverse Events (CTCAE) version 4.03 21. A modified grading system used to characterize TTFields-related skin adverse events is shown in Table 5.

iv. Study Endpoints

The primary endpoint was overall survival in patients receiving TTFields with standard therapy compared to standard therapy alone. Key secondary endpoints were overall survival in subgroups receiving docetaxel or an immune checkpoint inhibitor. Other secondary endpoints included progression-free survival and overall response rate (both per radiological assessment), overall survival and progression-free survival by squamous and non-squamous histology, and adverse events. All study endpoints are listed in Table 6.

Overall survival was defined as the time from randomization to date-of-death from any cause or censoring at the last follow-up date. Progression-free survival was defined as the time from the date of randomization until the date of disease progression in the liver or thorax, or death by any cause. Deaths that occurred without the investigator detecting tumor progression were considered progression events if they occurred within 6 weeks of the final tumor assessment prior to death. Otherwise, the patient was censored at the date of the final tumor assessment. Patients who had not progressed or died at the time of analysis were censored at the date of the most recent evaluable tumor assessment. Patients with no post-baseline tumor assessment were censored at the date of randomization. Overall radiological response rate was defined as a complete or partial response, and best response (categorized by complete response, partial response, stable disease, progressive disease, or not evaluable) was calculated for each treatment.

v. Statistical Analysis

The study was designed to detect a hazard ratio of <0.75 for overall survival in patients receiving TTFields with standard therapy versus standard therapy alone, using two-sided proportional hazards testing, a two-sided alpha of 0.05, and 80% power. This required a sample size of 534 patients, after allowing for 10% patient loss during follow-up, with an 18 month follow up. The key secondary endpoints of overall survival in the docetaxel and immune checkpoint inhibitor subgroups were to be tested hierarchically if the primary endpoint was met (to preserve type I error) at the 0.05 (two-sided) level.

Overall survival and progression-free survival were evaluated with two-sided log rank tests, at an alpha level of 0.05, stratified by treatment (immune checkpoint inhibitor or docetaxel), and tumor histology. Medians, confidence intervals (CIs), and rates were estimated using the Kaplan-Meier method. Hazard ratios with 95% CI and P-values were estimated using stratified Cox proportional hazards models with stratification variables introduced as covariates. For comparison of overall response rates, the 95% CI was calculated based on the exact binomial distribution (Clopper-Pearson). The difference between treatment arms, as well as the associated two-sided 95% CI, was calculated using a chi square test with significance at 0.05 (two-sided P value). Best responses were summarized per treatment group.

Efficacy endpoints were analyzed in all randomized patients (the intent-to-treat population). Safety and treatment data were compiled from all patients who received any study therapy and were analyzed according to the actual treatment received. Analyses were performed using SAS software, version 9.4 (SAS Institute). Full details are provided in the statistical analysis plan, available online with the article.

Following the pre-specified DMC annual review and interim analysis, the DMC concluded that continuing accrual to 534 patients was likely unnecessary and possibly unethical, and recommended that accrual of approximately 276 patients, with 12 months follow up, would provide toxicity and efficacy data to evaluate the planned study endpoints, while maintaining the statistical power. The DMC recommended no further changes to the study protocol.

### 2. Results

i. Patients and Treatments

Between February 2017 and November 2021, 276 patients were screened for eligibility and randomly assigned to receive TTFields with standard therapy (n=137) or standard therapy alone (n=139); the intent to treat population (FIG. 4).

Baseline demographics and characteristics were similar across groups (Table 1), and representative of patients with metastatic NSCLC (Table S5). Median age was 64.0 years (range, 22 to 86), 178 (65%) were male, and 232 (84.1%) were current or former smokers. The majority of patients (151 [56%]) had non-squamous NSCLC histology, 43 (16%) had liver metastasis, and 10 (3.6%) had an ECOG performance status of 2. Most patients (246 [89%]) had received one prior line of systemic therapy; patients in the docetaxel subgroup were more likely to have received prior treatment with an immune checkpoint inhibitor (82 patients [58%] vs 3 patients [2.2%]) (Table 1).

During the study, patients received a mean of 8.7 (range 1-70) cycles of standard therapy. The mean duration of TTFields therapy was 31.8 weeks (range 0.3 to 201) with an immune checkpoint inhibitor and 17.3 weeks (range 0.1 to 163) with docetaxel. A monthly average device usage of at least 18 hours/day was achieved by 13 patients (19.4%) with an immune checkpoint inhibitor and 17 patients (25.8%) with docetaxel.

Approximately 25% of patients received salvage systemic therapies following disease progression. The most frequently administered treatments were docetaxel (24 patients [8.7%]) and gemcitabine (21 patients [7.6%]) (Table 8).

ii. Efficacy

Treatment with TTFields plus a checkpoint inhibitor provides particularly improved results for patients with a diagnosis of metastatic NSCLC that had shown progression while on or after platinum-based systemic therapy, and whose prior systemic therapy had not included an ICI. Treatment with TTFields plus docetaxel provides particularly improved results for patients with a diagnosis of metastatic NSCLC that had shown progression while on or after systemic therapy which may have included an ICI.

a. Overall Survival

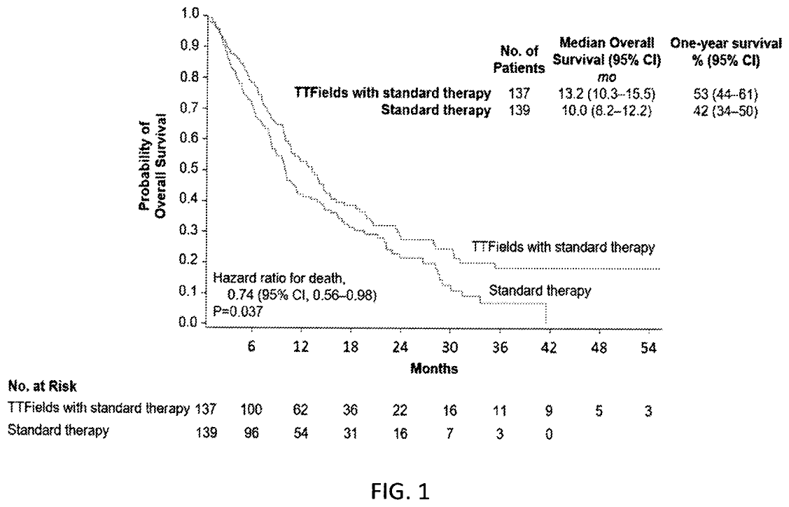

Overall survival was significantly longer with TTFields and standard therapy versus standard therapy alone (FIG. 1). After a minimum follow-up of 12 months, median overall survival was 13.2 months (95% confidence interval [CI], 13.3 to 15.5) with TTFields and standard therapy relative to 10.0 months (95% CI, 8.2 to 12.2) with standard therapy alone; hazard ratio of death, 0.74 (95% CI, 0.56 to 0.98; P=0.037). The one-year overall survival rate was 53% (95% CI, 44 to 61) with TTFields and standard therapy, and 42% (95% CI, 34 to 50) with standard therapy alone (Table 2).

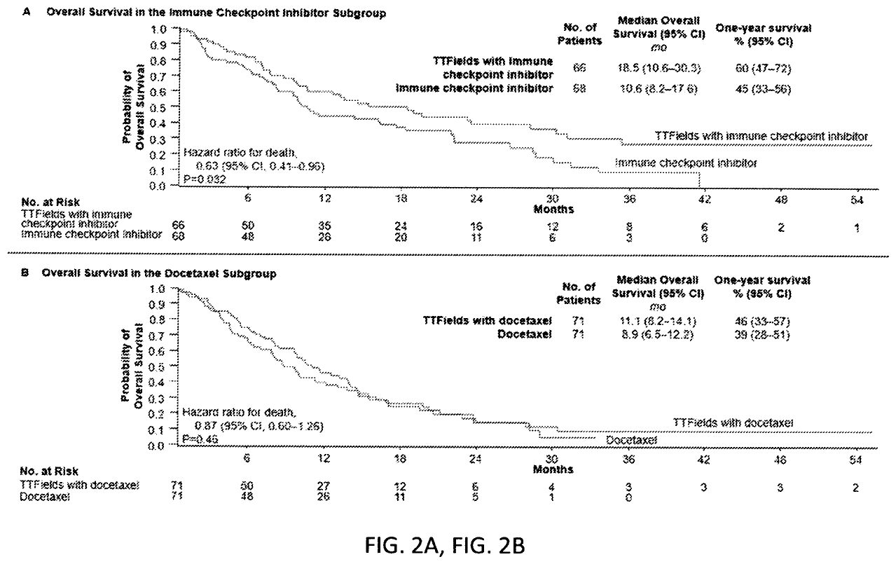

In patients receiving an immune checkpoint inhibitor, TTFields therapy significantly improved overall survival compared to an immune checkpoint inhibitor alone. Median overall survival was 18.5 months (95% CI, 10.6 to 30.3) versus 10.6 months (95% CI, 8.2 to 17.6) and hazard ratio of death, 0.63 (95% CI, 0.41 to 0.96; P=0.032) (FIG. 2A). The survival rate at one-year was 60% (95% CI, 47 to 72) with TTFields therapy and an immune checkpoint inhibitor, and 45% (95% CI 33 to 56) with an immune checkpoint inhibitor alone. The clinical evidence disclosed herein suggests that applying TTFields with ICI increases tumor control and enhances antitumor immunity. TTFields have been demonstrated to be additive or synergistic when combined with different systemic therapies including but not limited to platinum agents, taxanes, pemetrexed and ICI. When added to ICI this was also accompanied by significantly higher expression of markers related to immunogenic cell death, as well as by higher infiltration of antigen presenting cells into the tumor in vivo. In the LUNAR study, concomitant therapy of TTFields and ICI significantly increased the median OS from 10.8 to 18.5 months comparing to ICI alone. These findings suggest that TTFields may augment the immune response against the tumor and reduce tolerance induced by tumor cells through PD-1/PD-L1 interactions.

In the subgroup receiving docetaxel, median overall survival was 11.1 months (95% CI, 8.2 to 14.1) with TTFields therapy (n=71) and 8.9 months (95% CI, 6.5 to 12.2) with docetaxel alone (n=71), with hazard ratio of death, 0.87; 95% CI, 0.60 to 1.26; P=0.46) (FIG. 2B). One-year survival rates were 45.6% (33.3-57.1) and 39.4% (28.1-50.5), respectively.

Patients with non-squamous disease receiving TTFields with standard therapy vs standard therapy alone had median overall survival of 12.6 months (95% CI 8.8-19.8) vs 9.9 months (95% CI 6.9-16.4). Respectively, patients with squamous disease and receiving TTFields had median overall survival of 13.9 months (95% CI 9.7-17.1) vs 10.1 months (95% CI 8.3-13.8) with standard therapy alone (FIG. S4).

b. Progression Free Survival and Overall Response

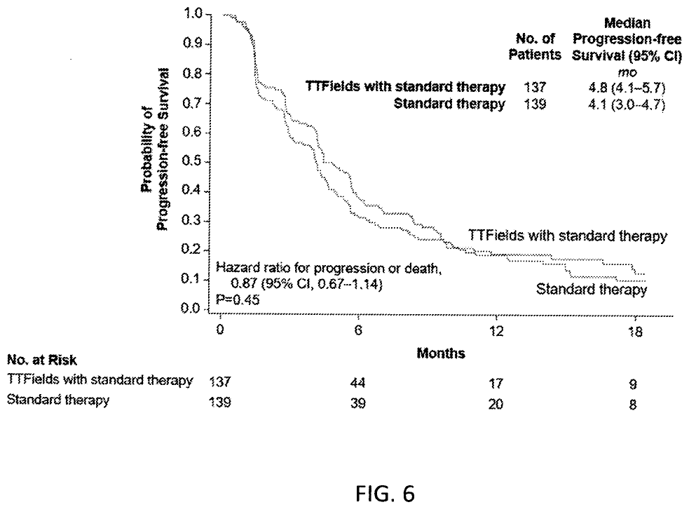

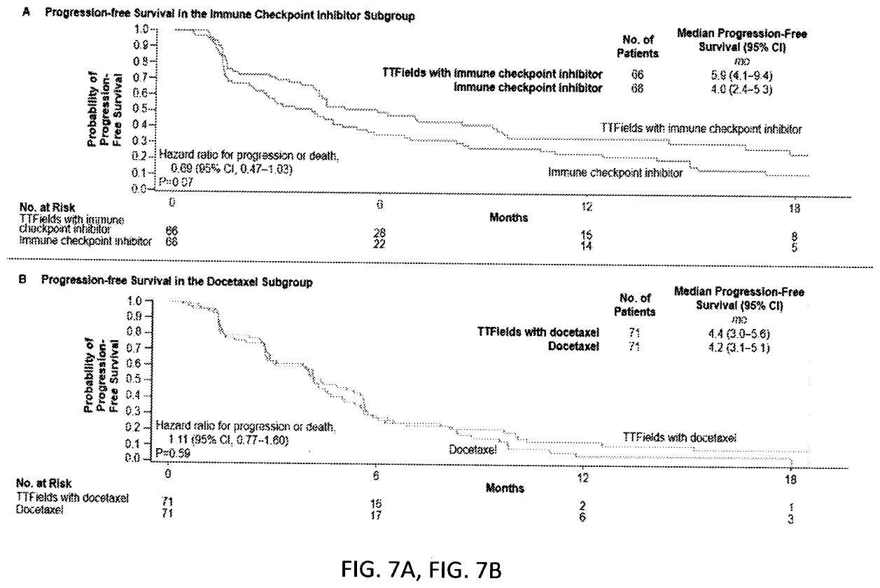

Median progression free survival was 4.8 months (95% CI, 4.1 to 5.7) with TTFields and standard therapy and 4.1 months (95% CI, 3.0 to 4.7) with standard therapy alone; hazard ratio of progression or death, 0.87 (95% CI, 0.67 to 1.14; P=0.45) (FIG. 6). Progression free survival in subgroups receiving an immune checkpoint inhibitor or docetaxel is shown in FIG. 7.

The overall response rate with TTFields and standard therapy was 20.4% (95% CI, 14.0 to 28.2), and with standard therapy alone was 17.3% (95% CI, 11.4 to 24.6) (Table 2). All complete responses (n=5) were in patients receiving an immune checkpoint inhibitor (n=4 with TTFields, n=1 with immune checkpoint inhibitor alone).

iii. Safety

In the safety population (267 patients), almost all (251 [94%]) reported at least one adverse event. In patients receiving TTFields with standard therapy versus standard therapy alone, frequencies were similar for adverse events of any grade (129 [97%] and 122 [91%]; Table 3) and lower for adverse events of grade 3-5 (69 [52%] and 84 [63%]; Table 9). Serious adverse events were reported by 70 (52.6%) patients with TTFields plus standard therapy and 51 (38.1%) with standard therapy alone. The most frequently reported adverse events were associated with the systemic therapies or the disease itself: anemia (n=59; 22.1%), dyspnea (n=57; 21.3%), fatigue (n=51; 29.1%), diarrhea (n=50; 18.7%) and nausea (n=45; 16.9%).

Most (71.4%) TTFields-treated patients had at least one device-related adverse event; eight (6%) were grade 3. There were no grade 4 toxicities and no deaths attributable to TTFields (Table S8). The most frequent TTFields-related adverse events were grade 1-2 skin adverse events: dermatitis (n=16 [12%]), skin irritation (15 [11.3%]), pruritus (13 [9.8%]), and rash (11 [8.3%]). The incidence of TTFields-related adverse events was generally comparable between treatment subgroups. The frequency of cardiac events was similar between patients receiving TTFields with standard therapy or standard therapy alone (19 and 18 respectively). No cardiac events were deemed related to TTFields therapy.

No patients discontinued TTFields therapy due to intolerable toxicity, versus two patients receiving immune checkpoint inhibitors and four patients receiving docetaxel.

### 3. Discussion

Since the adoption of immune checkpoint inhibitors as standard of care, no additional phase 3 studies have demonstrated improvement in the survival of patients with metastatic NSCLC after failure of platinum therapy. Given the high prevalence of NSCLC, and that most patients with metastatic disease progress on first line therapy, there is a pressing need for additional, effective, and tolerable treatment options in this setting. The randomized, phase 3 LUNAR study demonstrated that TTFields therapy, an innovative locoregional treatment modality, applied concomitantly with systemic standard of care significantly improved overall survival in patients with metastatic NSCLC following platinum failure, compared to standard systemic therapy alone. The overall survival benefit with TTFields therapy occurred without increasing the systemic toxicities associated with standard of care, and with a safety profile mostly limited to low grade skin adverse events.

Platinum therapy remains a frequently administered standard of care mainstay in NSCLC; in combination with immune checkpoint inhibitors (first line therapy), or after patients progress on immune checkpoint inhibitor monotherapy (second line therapy). Optimal treatment following progression on platinum-based therapy is still an unmet need. The greater than three-month improvement in overall survival with the addition of TTFields is clinically meaningful, substantiating use in this highly burdened patient population with limited treatment options. Indeed, a survival benefit of this magnitude is consistent with survival improvements reported from the landmark studies of immune checkpoint inhibitors (versus docetaxel) that established the current standard of care in second line advanced NSCLC. The survival benefit with addition of TTFields was also comparable to that reported in a recent phase 2 study that evaluated combination pembrolizumab and ramucirumab (14.5 months) vs standard of care (11.6 months) in patients who progressed on immune checkpoint inhibitor plus platinum therapy.

Interestingly, the comparable progression free survival between groups observed here is consistent with results from several immunotherapy studies in NSCLC. This outcome may be explained by a delayed tumor response to therapy and a longer survival post-progression with immunotherapies relative to cytotoxic chemotherapy.

TTFields therapy had a profound eight-month survival benefit in the subgroup receiving an immune checkpoint inhibitor. These results are underscored by findings in preclinical lung cancer models, where TTFields treatment induced immunogenic cell death, priming an anticancer immune response that could then be sustained via treatment with immune checkpoint inhibitors, leading to enhanced effectiveness when both modalities were used together. Although the strong efficacy benefit of TTFields therapy in the immune checkpoint inhibitor subgroup was not matched in the subgroup receiving docetaxel, it is notable that patients in the docetaxel subgroup had a lower median duration of TTFields therapy than the immune checkpoint inhibitor subgroup and that about half of these patients were previously treated with an immune checkpoint inhibitor. Additionally, the absence of statistically significant benefit in this subgroup may be influenced by the fact that LUNAR was only powered to detect efficacy benefits in the intent to treat population.

The TTFields therapy safety profile in LUNAR was limited to mild-to-moderate local skin irritation underneath the arrays, with no evidence of internal or systemic safety concerns, including cardiac events. These data are consistent with previous clinical and real world studies of TTFields therapy in other tumor types. The skin adverse events related to TTFields therapy normally arise from contact with the adhesive or hydrogel on the arrays, and are not due to the therapy itself. In most cases skin adverse events are effectively controlled using prophylaxis and topical therapies, and increased patient/caregiver education may reduce the risk of their development. While the analysis of quality of life data from LUNAR is ongoing, patient-reported outcomes reported in newly diagnosed glioblastoma studies show that the device did not impair quality of life; indeed, TTFields therapy postponed the decline in quality of life compared to patients receiving standard of care alone.

Limitations of the LUNAR study include its open-label study design, which was considered appropriate based on the practical challenge and ethical concerns of exposing patients to a sham device. The study enrolled a low number of patients with brain metastases, potentially affecting the generalizability of these findings to that population. LUNAR was initiated before next-generation sequencing genetic profiling was standard in NSCLC, and thus only limited information about the relationship between TTFields therapy efficacy and genetic subtypes of tumor is available. Nevertheless, the study was open to a broad population with no restrictions on tumor biomarker or histological status, or type of prior therapy beyond progression of platinum.

Overall, the LUNAR study showed that TTFields therapy significantly improved overall survival when added to standard of care systemic therapies for patients with metastatic NSCLC progressing on or after platinum therapy. There were no new safety signals, and TTFields therapy did not exacerbate systemic toxicities of immune checkpoint inhibitors or docetaxel. These phase 3 efficacy and safety data support TTFields therapy as part of second-line standard of care in metastatic NSCLC.

Those skilled in the art will recognize, or be able to ascertain using no more than routine experimentation, many equivalents to the specific embodiments of the method and compositions described herein. Such equivalents are intended to be encompassed by the claims below.

TABLE 1
Patient Demographics and Baseline Disease Characteristics

| | | | TTFields | | | |
|---|---|---|---|---|---|---|
| | TTFields | | Therapy | | | |
| | Therapy | | With an | | TTFields | |
| | With | | Immune | Immune | Therapy | |
| | Standard | Standard | Checkpoint | Checkpoint | With | |
| | Therapy | Therapy | Inhibitor | Inhibitor | Docetaxel | Docetaxel |
| | N = 137 | N = 139 | N = 66 | N = 68 | N = 71 | N = 71 |
| Age - yr | | | | | | |
| Median | 63.0 | 65.0 | 64.0 | 65.0 | 63.0 | 65.0 |
| Range | (36, 85) | (22, 86) | (36, 85) | (23, 86) | (43, 81) | (22, 81) |
| Male sex - no. (%) | 91 (66.4) | 87 (62.6) | 44 (66.7) | 45 (66.2) | 47 (66.2) | 42 (59.2) |
| Race - no. (%) | | | | | | |
| American Indian or | 0 | 2 (1.4) | 0 | 1 (1.5) | 0 | 1 (1.5) |
| Alaska Native | | | | | | |
| Asian | 16 (11.7) | 12 (8.6) | 7 (10.6) | 5 (7.4) | 9 (12.7) | 7 (9.9) |
| Black or African | 3 (2.2) | 3 (2.2) | 1 (1.5) | 2 (2.9) | 2 (2.8) | 1 (1.4) |
| American | | | | | | |
| Pacific Islander | 1 (0.7) | 0 | 1 (1.5) | 0 | 0 | 0 |
| White | 111 (81.0) | 111 (79.9) | 54 (81.8) | 53 (77.9) | 57 (80.3) | 58 (81.7) |
| Other | 5 (3.6) | 8 (5.8) | 3 (4.5) | 4 (5.9) | 2 (2.8) | 4 (5.6) |
| Region- no. (%) | | | | | | |
| North America | 41 (29.9) | 43 (30.9) | 14 (21.2) | 17 (25.0) | 27 (38.0) | 26 (36.6) |
| Western Europe | 42 (30.7) | 41 (29.5) | 25 (37.9) | 24 (35.3) | 17 (23.9) | 17 (23.9) |
| and Israel | | | | | | |
| Eastern Europe | 41 (29.9) | 43 (30.9) | 21 (31.8) | 22 (32.4) | 20 (28.2) | 21 (29.6) |
| East Asia | 13 (9.5) | 12 (8.6) | 6 (9.1) | 5 (7.4) | 7 (9.9) | 7 (9.9) |
| ECOG performance | | | | | | |
| status score - | | | | | | |
| no. (%) | | | | | | |
| 0 | 37 (27.0) | 40 (28.8) | 19 (28.8) | 22 (32.4) | 18 (25.4) | 18 (25.4) |
| 1 | 92 (67.2) | 95 (68.3) | 43 (65.2) | 46 (67.6) | 49 (69.0) | 49 (69.0) |
| 2 | 6 (4.4) | 4 (2.9) | 2 (3.0) | 0 | 4 (5.6) | 4 (5.6) |
| Missing | 2 (1.5) | 0 | 2 (3.0) | 0 | 0 | 0 |
| Smoking history - | | | | | | |
| no. (%) | | | | | | |
| Never smoked | 20 (14.6) | 23 (16.5) | 10 (15.2) | 12 (17.6) | 10 (14.1) | 11 (15.5) |
| Current smoker | 35 (25.5) | 29 (20.9) | 19 (28.8) | 17 (25.0) | 16 (22.5) | 12 (16.9) |
| Former smoker | 81 (59.1) | 87 (62.6) | 37 (56.1) | 39 (57.4) | 44 (62.0) | 48 (67.6) |
| Unknown | 1 (0.7) | 0 | 0 | 0 | 1 (1.4) | 0 |
| Time - mo since | | | | | | |
| initial NSCLC | | | | | | |
| diagnosis | | | | | | |
| Median | 10.3 | 9.9 | 10.1 | 8.5 | 10.4 | 11.1 |
| Range | (2.7-127.2) | (2.5-164.6) | (2.8-98.4) | (2.7-164.6) | (2.7-127.2) | (2.5-68.9) |
| Any Prior Therapy | | | | | | |
| for NSCLC, n (%) | | | | | | |
| Yes | 137 (100) | 139 (100) | 66 (100) | 67 (98.5) | 71 (100) | 71 (100) |
| If Yes, Best | | | | | | |
| Response to Prior | | | | | | |
| Therapy | | | | | | |
| Complete Response | 8 (5.8) | 5 (3.6) | 4 (6.1) | 3 (4.5) | 4 (5.6) | 2 (2.8) |
| Partial Response | 32 (23.4) | 36 (25.9) | 19 (28.8) | 13 (19.4) | 13 (18.3) | 23 (32.4) |
| Stable Disease | 47 (34.3) | 44 (31.7) | 25 (37.9) | 21 (31.3) | 22 (31.0) | 23 (32.4) |
| Progressive Disease | 29 (21.2) | 36 (25.9) | 10 (15.2) | 20 (29.9) | 19 (26.8) | 16 (22.5) |
| Unknown | 21 (15.3) | 17 (12.2) | 8 (12.1) | 10 (14.9) | 13 (18.3) | 7 (9.9) |
| Missing | 0 | 1 (0.7) | 0 | 1 (1.5) | 0 | 0 |
| No | 0 | 0 | 0 | 1 (1.5) | 0 | 0 |
| Previous lines of | | | | | | |
| systemic therapy - | | | | | | |
| no. (%) | | | | | | |
| 1 | 122 (89.1) | 124 (89.2) | 64 (97) | 64 (94.1) | 58 (81.7) | 60 (84.5) |
| 2 | 9 (6.6) | 12 (8.6) | 2 (3) | 3 (4.4) | 7 (9.9) | 9 (12.7) |
| 3+ | 6 (4.4) | 2 (1.4) | 0 | 0 | 6 (8.5) | 2 (2.8) |
| Prior immune | | | | | | |
| checkpoint | | | | | | |
| inhibitor - | | | | | | |
| no. (%) | | | | | | |
| Yes | 42 (30.7) | 43 (30.9) | 1 (1.5) | 2 (2.9) | 41 (57.7) | 41 (57.7) |
| No | 95 (69.3) | 96 (69.1) | 65 (98.5) | 66 (97.1) | 30 (42.3) | 30 (42.3) |
| Prior immune | | | | | | |
| checkpoint | | | | | | |
| inhibitor as | | | | | | |
| only prior | | | | | | |
| therapy - | | | | | | |
| no. (%) | | | | | | |
| Yes | 26 (61.9) | 30 (69.8) | 0 | 1 (50) | 26 (63.4) | 29 (70.7) |
| No | 16 (38.1) | 13 (30.2) | 1 (100.0) | 1 (50) | 15 (36.6) | 12 (29.2) |
| Histological | | | | | | |
| type - no. (%) | | | | | | |
| Non-squamous | 79 (57.7) | 76 (54.7) | 37 (56.1) | 36 (52.9) | 42 (59.2) | 40 (56.3) |
| Squamous | 58 (42.3) | 63 (45.3) | 29 (43.9) | 32 (47.1) | 29 (40.8) | 31 (43.7) |
| Programmed cell | | | | | | |
| death ligand 1 | | | | | | |
| tumor proportion | | | | | | |
| score - no. (%) | | | | | | |
| <1% | 23 (16.8) | 23 (16.5) | 12 (18.2) | 16 (23.5) | 11 (15.5) | 7 (9.9) |
| 1-49% | 37 (27.0) | 40 (28.8) | 17 (25.8) | 18 (26.5) | 20 (28.2) | 22 (31.0) |
| ≥50% | 10 (7.3) | 18 (12.9) | 5 (7.6) | 8 (11.8) | 5 (7.0) | 10 (14.1) |
| Unknown | TBC | TBC | TBC | TBC | TBC | TBC |
| Liver | | | | | | |
| metastasis - | | | | | | |
| no. (%) | | | | | | |
| Yes | 21 (15.3) | 22 (15.8) | 9 (13.6) | 8 (11.8) | 12 (16.9) | 14 (19.7)^{†} |
| No | 116 (84.7) | 117 (84.2) | 57 (86.4) | 60 (88.2) | 59 (83.1) | 57 (80.3) |
| Central nervous | | | | | | |
| system | | | | | | |
| metastasis- | | | | | | |
| no. (%)^{‡} | | | | | | |
| Yes | 0 | 2 (1.4) | 0 | 0 | 0 | 2 (2.8)^{†} |
| No | 137 (100) | 137 (98.6) | 66 (100) | 68 (100) | 71 (100) | 69 (97.1) |

*Radiological progress per RECIST v1.1.
^{†}One patient had both liver and CNS metastasis.
^{‡}Patients with central nervous system metastases were excluded under the original study design that was later amended to allow enrollment of patients with stable metastases.

TABLE 2
Response rates.

| | TTFields With | Standard |
|---|---|---|
| | Standard Therapy | Therapy |
| No. of patients | 137 | 139 |
| Overall response rate-no. (%), | 28 (20.4) | 24 (17.3) |
| [95% CI] | [14.0-28.2] | [11.4; 24.6] |
| Best overall response-no. (%) | | |
| Complete response | 4 (2.9) | 1 (0.7) |
| Partial response | 24 (17.5) | 23 (16.5) |
| Stable disease | 67 (48.9) | 65 (46.8) |
| Progressive disease | 24 (17.5) | 36 (25.9) |
| Not evaluable | 3 (2.2) | 2 (1.4) |

TABLE 3
Adverse Events.

| | TTFields Therapy | | | | | | | |
|---|---|---|---|---|---|---|---|---|
| | With an Immune | | Immune | | TTFields | | | |
| | Checkpoint | | Checkpoint | | Therapy | | | |
| | Inhibitor | | Inhibitor | | With docetaxel | | Docetaxel | |
| | N = 67 | | N = 66 | | N = 66 | | N = 68 | |
| Preferred term - | All | | All | | All | | All | |
| no. (%) | grades | Grade ≥3 | grades | Grade ≥3 | grades | Grade ≥3 | grades | Grade ≥3 |
| Any adverse event | 66 (98.5) | 37 (55.2) | 61 (92.4) | 32 (48.5) | 63 (95.5) | 41 (62.1) | 61 (89.7) | 43 (63.2) |
| Serious adverse | 34 (50.7) | | 23 (34.8) | | 36 (54.5) | | 28 (41.2) | |
| event | | | | | | | | |
| Adverse event | 4 (5.9) | | 1 (1.5) | | 4 (6.1) | | 1 (1.5) | |
| leading to | | | | | | | | |
| discontinuation | | | | | | | | |
| Adverse event | 5 (7.5) | | 6 (9.1) | | 8 (12.1) | | 4 (5.9) | |
| leading to death | | | | | | | | |
| Adverse events* | | | | | | | | |
| Blood and lymphatic | | | | | | | | |
| system disorders | | | | | | | | |
| Anemia | 17 (25.4) | 5 (7.5) | 9 (13.6) | 2 (3.0) | 14 (21.2) | 5 (7.6) | 19 (27.9) | 8 (11.8) |
| Neutropenia | 2 (3.0) | 2 (3.0) | 3 (4.5) | 1 (1.5) | 8 (12.1) | 7 (10.6) | 15 (22.1) | 11 (16.2) |
| Gastrointestinal | | | | | | | | |
| disorders | | | | | | | | |
| Diarrhea | 12 (17.9) | TBC | 13 (19.7) | TBC | 13 (19.7) | TBC | 12 (17.6) | TBC |
| Nausea | 7 (10.4) | TBC | 11 (16.7) | TBC | 17 (25.8) | TBC | 10 (14.7) | TBC |
| Constipation | 4 (6.0) | TBC | 5 (7.6) | TBC | 11 (16.7) | TBC | 10 (14.7) | TBC |
| Vomiting | 6 (9.0) | TBC | 7 (10.6) | TBC | 8 (12.1) | TBC | 7 (10.3) | TBC |
| Dysphagia | 4 (6.0) | TBC | 1 (1.5) | TBC | 9 (13.6) | TBC | 1 (1.5) | TBC |
| General disorders | | | | | | | | |
| and administration | | | | | | | | |
| site conditions | | | | | | | | |
| Fatigue | 6 (9.0) | TBC | 11 (16.7) | TBC | 16 (24.2) | TBC | 18 (26.5) | TBC |
| Asthenia | 11 (16.4) | TBC | 12 (18.2) | TBC | 6 (9.1) | TBC | 10 (14.7) | TBC |
| Oedema | 5 (7.5) | TBC | 7 (10.6) | TBC | 10 (15.2) | TBC | 9 (13.2) | TBC |
| peripheral | | | | | | | | |
| Pyrexia | 5 (7.5) | TBC | 9 (13.6) | TBC | 4 (6.1) | TBC | 9 (13.2) | TBC |
| Infections and | | | | | | | | |
| infestations | | | | | | | | |
| Pneumonia | 8 (11.9) | TBC | 9 (13.6) | TBC | 10 (15.2) | TBC | 11 (16.2) | TBC |
| Upper respiratory | 3 (4.5) | TBC | 8 (12.1) | TBC | 1 (1.5) | TBC | 1 (1.5) | TBC |
| tract infection | | | | | | | | |
| Investigations | | | | | | | | |
| White blood cell | 1 (1.5) | TBC | 1 (1.5) | TBC | 8 (12.1) | TBC | 7 (10.3) | TBC |
| count decreased | | | | | | | | |
| Neutrophil count | 1 (1.5) | TBC | 1 (1.5) | TBC | 8 (12.1) | TBC | 6 (8.8) | TBC |
| decreased | | | | | | | | |
| Metabolism and | | | | | | | | |
| nutrition disorders | | | | | | | | |
| Decreased | 11 (16.4) | TBC | 8 (12.1) | TBC | 11 (16.7) | TBC | 11 (16.2) | TBC |
| appetite | | | | | | | | |
| Hypoalbuminemia | 3 (4.5) | TBC | 9 (13.6) | TBC | 7 (10.6) | TBC | 8 (11.8) | TBC |
| Hyponatremia | 5 (7.5) | TBC | 9 (13.6) | TBC | 7 (10.6) | TBC | 4 (5.9) | TBC |
| Hypokalemia | 4 (6.1) | TBC | 4 (6.0) | TBC | 8 (12.1) | TBC | 8 (11.8) | TBC |
| Musculoskeletal and | | | | | | | | |
| connective tissue | | | | | | | | |
| disorders | | | | | | | | |
| Back pain | 9 (13.4) | TBC | 9 (13.6) | TBC | 10 (15.2) | TBC | 4 (5.9) | TBC |
| Arthralgia | 3 (4.5) | TBC | 6 (9.1) | TBC | 5 (7.6) | TBC | 7 (10.3) | TBC |
| Nervous system | | | | | | | | |
| disorders | | | | | | | | |
| Headache | 8 (11.9) | TBC | 5 (7.6) | TBC | 4 (6.1) | TBC | 6 (8.8) | TBC |
| Respiratory, | | | | | | | | |
| thoracic and | | | | | | | | |
| mediastinal | | | | | | | | |
| disorders | | | | | | | | |
| Dyspnea | 10 (14.9) | TBC | 13 (19.7) | TBC | 15 (22.7) | TBC | 19 (27.9) | TBC |
| Cough | 8 (11.9) | TBC | 14 (21.2) | TBC | 10 (15.2) | TBC | 11 (16.2) | TBC |
| Pleural effusion | 4 (6.0) | TBC | 2 (3.0) | TBC | 3 (4.5) | TBC | 8 (11.8) | TBC |
| Skin and | | | | | | | | |
| subcutaneous | | | | | | | | |
| tissue | | | | | | | | |
| disorders | | | | | | | | |
| Alopecia | 0 | TBC | 1 (1.5) | TBC | 13 (19.7) | TBC | 22 (32.4) | TBC |
| Pruritus | 9 (13.4) | TBC | 7 (10.6) | TBC | 11 (16.7) | TBC | 0 | TBC |
| Rash | 9 (13.4) | TBC | 1 (1.5) | TBC | 7 (10.6) | TBC | 2 (2.9) | TBC |
| Skin irritation | 10 (14.9) | TBC | 0 | TBC | 7 (10.6) | TBC | 0 | TBC |
| Dermatitis | 9 (13.4) | TBC | 0 | TBC | 7 (10.6) | TBC | 0 | TBC |
| Rash | 2 (3.0) | TBC | 2 (3.0) | TBC | 8 (12.1) | TBC | 3 (4.4) | TBC |
| maculopapular | | | | | | | | |

Adverse events were compiled from the safety population.
*10% of patients in any arm/group

### 4. Supplemental Methods

### 1. Follow-Up Visits

Follow-up visits were conducted every 6 weeks (±1 week) for radiological assessment of disease per response evaluation criteria in solid tumors (RECIST) criteria for patients receiving docetaxel, or immune-related (ir)RECIST for patients receiving an immune checkpoint inhibitor. Visits also included computed tomography, positron emission tomography, or magnetic resonance imaging scans, review of TTFields therapy usage time, adverse event reporting, physical examination, and laboratory testing. After one year of follow-up, imaging examinations were performed once every 12 weeks. Further follow-up to monitor survival was carried out every 4 weeks (±1 week) during a visit to the study center or telephone call. After progression in the thorax and/or liver, additional follow-up visits took place 30, 60, and 100 days (all+1 week) after disease progression.

### 2. Concomitant Medication

During treatment, a high potency topical steroid (e.g. clobetasol) or antibiotic ointment could be prescribed and applied by the patient/caregiver (after removing the arrays and cleaning skin with baby oil and medical alcohol) if skin irritation/abrasion occurred with TTFields therapy.

TABLE 4
Study Inclusion and Exclusion Criteria.

```
Inclusion criteria
  22 years of age and older
  Life expectancy greater than three months
  ECOG performance status of 0-2
  Histological or cytological diagnosis of squamous or
  non-squamous, inoperable, metastatic NSCLC made
  while on or after first platinum-based systemic therapy
  administered for advanced or metastatic disease.
    Patients who developed metastatic disease within 6
    months of completing adjuvant or neoadjuvant
    platinum-based chemotherapy (after surgery and/or
    radiation therapy) are also eligible. If longer
    than 6 months, then the patient must have also
    received platinum-based therapy in the advanced
    setting.
    Platinum-based therapy must have been the most
    recent therapy prior to enrollment, aside from a
    maintenance therapy.
  Able to operate the NovoTTF-200T device
  independently or with the help of a caregiver.
  Provided signed informed consent.
Exclusion criteria
  Metastases to CNS with clinical symptoms or evidence
  of new CNS metastases during screening. Note
  that patients with previously treated CNS metastases,
  that are stable and meet the following requirements
  are eligible:
    Patients were neurologically returned to baseline
    (except for residual signs or symptoms related to
    CNS treatment).
    Patients received no treatment (i.e. surgery,
    radiotherapy, corticosteroid therapy) for CNS
    metastases during the screening period.
    CNS lesions did not progress (by MRI)
    within 14 days prior to randomization.
    There was no meningeal metastasis or
    spinal cord compression.
  Patients due to receive an immune checkpoint inhibitor
  had no contraindications to receive an immune
  checkpoint inhibitor.
  Patients due to receive docetaxel had no
  contraindications to receive docetaxel.
  Severe comorbidities:
    Clinically significant (as determined by the
    investigator) hematological, hepatic or renal
    dysfunction, defined as: Neutrophil count <1.5 x
    10 9/L and platelet count <100 × 10 9/L;
    bilirubin >1.5 × ULN; AST and/or ALT >2.5 ×
    ULN or >5 × ULN if patient has documented
    liver metastases; and serum creatinine >1.5 × ULN.
    History of significant cardiovascular disease
    unless the disease is well controlled. Including,
      Second/third degree heart block.
      Significant ischemic.
      Poorly controlled hypertension.
      Congestive heart failure of the New York
      Heart Association Class II or worse (slight
      limitation of physical activity; comfortable
      at rest, but ordinary activity results in fatigue,
      palpitation or dyspnea).
      History of arrhythmia that is symptomatic
      or requires treatment. Patients with atrial
      fibrillation or flutter controlled by
      medication are not excluded.
      History of pericarditis.
    History of interstitial lung disease.
    A cerebrovascular accident within 6 moths
    prior to randomization or that is unstable
    Active infection or serious underlying medical
    condition that would impair the ability of the
    patient to receive study therapy.
    History of any psychiatric condition that might
    impair understanding or compliance with the study or
    consent.
    Any other malignancy requiring anti-tumor t
    reatment in the past three years, excluding treated
    stage I prostate cancer, in situ cervical cancer,
    in situ breast cancer and non-melanomatous skin
    cancer
  Concurrent treatment with another experimental
  NSCLC treatment while in the study.
  Implanted electronic medical device (e.g.
  pacemaker, defibrillator) in the upper torso.
  Known allergies to medical adhesives or hydrogel.
  Pregnancy or breast-feeding (patients with reproductive
  potential must use effective contraception
  methods throughout the entire study period, as
  determined by their investigator/gynecologist).
  Admitted to an institution by administrative or court order.
ALT, alanine aminotransferase;
AST, Aspartate Aminotransferase;
CNS, central nervous system;
ECOG, Eastern Cooperative Oncology Group;
ICI, immune checkpoint inhibitor;
NSCLC, non-small cell lung cancer;
ULN, upper limit of normal
```

TABLE 5
Grading for TTFields Device-Related Skin Adverse Events

Grade 1
Asymptomatic or mild symptoms AND
1. No intervention required OR only topical
treatment intervention indicated
2. Treatment interruption of less than 3 days may be required
Grade 2
Moderate symptoms AND Systemic therapy required
OR event is requiring interruption of TTFields
for more than 3 days
Grade 3
Severe or medically significant but not immediately
life threatening AND hospitalization OR
prolongation of existing hospitalization indicated
Grade 4
Life threatening consequences AND urgent intervention indicated

TABLE 6
Study End Points

```
Primary endpoint
  Overall survival in the overall population
Key secondary endpoints
  Overall survival of patients treated with an immune checkpoint inhibitor
  Overall survival of patients treated with docetaxel
Additional secondary endpoints
  Overall survival of patients who received TTFields
  therapy with docetaxel compared to patients
  treated with an immune checkpoint inhibitor alone
  Progression-free survival in the overall population
  Overall radiological response rate in the overall population.
  Additionally;
    number of patients with complete response,
    partial response, stable disease, progressive
    disease (and not evaluable response) for each treatment
    overall response of target lesions, non-target
    lesions, and new lesions (by treatment at each
    visit).
  Quality of life in the overall population
  Overall survival and progression-free survival
  in subgroups who received nivolumab,
  pembrolizumab, or atezolizumab
  Overall survival and progression-free survival in
  subgroups with squamous and non-squamous
  histology
  Overall survival and progression-free survival in
  TTFields therapy-treated subgroups with average
  monthly device usage >75 and ≤75%
  Safety in patients who received any amount of
  TTFields therapy or standard therapy alone during the
  study
```

Efficacy endpoints were measured in the intent-to-treat population that consisted of all randomized subjects regardless of treatment receipt. Overall survival was measured as time from randomization to date-of-death of any cause (or censoring at the most recent follow-up date). Progression-free survival was defined as time from randomization to date of disease progression in the liver or thorax (according to RECIST/irRECIST), or date of death of any cause, or (if no progression or death) censoring at the date of the most recent evaluable RECIST/irRECIST assessment (or randomization in the event of no evaluable post-baseline tumor assessment). Deaths that occurred within 6 weeks of a tumor assessment without progression were considered as an event. Otherwise, the patient was censored at the most recent tumor assessment. Radiological response was defined as patients whose best overall response was a partial response or a complete response.

ICI, immune-checkpoint inhibitor; SOC, standard of care; TTFields, Tumor Treating Fields

TABLE 7
Representativeness of the Study Population

| Category | |
|---|---|
| Disease, problem, or | Metastatic non-small cell lung cancer |
| condition under | |
| investigation | |

Special considerations related to

| | |
|---|---|
| Sex and gender | Lung cancer is slightly more frequent |
| | (by 24%) in men than women in |
| | the USA (Siegel et al 2022), while |
| | occurs in around twice as many |
| | men than women in the EU and |
| | China (Zhou 2014; Cao et al 2019; |
| | OECD et al 2022). |
| Age | Most lung cancer is diagnosed in |
| | individuals aged ≥65 years (US |
| | National Cancer Institute ; Zhou 2014) |
| Race or ethnic group | In the US, most individuals diagnosed |
| | with NSCLC were White |
| | (76%), 11% were Black, 7% were |
| | Hispanic, 6% were Asian/Pacific |
| | Islander, and 0.3% were Native |
| | American (US National Cancer |
| | Institute). |
| Geographical region | Men in Eastern Europe and Eastern |
| | Asia show the highest incidence |
| | of lung cancer (Cheng et al 2016). |
| | In general, the incidence of lung |
| | cancer is highest in countries with |
| | the highest socioeconomic |
| | development (Cheng et al 2016). |

Other considerations

| | |
|---|---|
| Smoking history | Over 80% of lung cancer cases are |
| | linked to smoking (Cao et al 2019; |
| | Siegel et al 2022). |
| Histological status | The relative frequency of different |
| | histological subtypes varies by |
| | country according to regional differences |
| | in smoking habits (Cheng et |
| | al 2016). In general, non-squamous |
| | NSCLC is more prevalent than |
| | squamous cell NSCLC (Cheng |
| | et al 2016), including it is |
| | approximately twice as common |
| | in the US (US National Cancer |
| | Institute) |
| Overall | The LUNAR patient characteristics |
| representativeness | of median 64 year age, 65% male, |
| of this trial | 80% White, 56%/44% non-squamous/ |
| | squamous histology, and 84% |
| | current or former smokers, is similar |
| | to the populations of other |
| | clinical trials in patients with |
| | advanced or metastatic NSCLC that had |
| | progressed on first line therapy |
| | (Borghaei et al 2015; Rittmeyer et al |
| | 2017; Reckamp et al 2022). LUNAR |
| | included patients recruited from |
| | Eastern Europe (30%) and East Asia |
| | (9%) that have high prevalences |
| | of NSCLC. |

TABLE 8
Treatments Received by Patients following Progression on Study Treatment

| | TTFields | | TTFields | | | |
|---|---|---|---|---|---|---|
| | Therapy | | Therapy with | | TTFields | |
| | with | | an Immune | Immune | Therapy | |
| | Standard | Standard | Checkpoint | Checkpoint | with | |
| | Therapy | Therapy | Inhibitor | Inhibitor | Docetaxel | Docetaxel |
| No. patients | 137 | 139 | 66 | 68 | 71 | 71 |
| randomized | | | | | | |
| Systemic salvage | 41 (29.9) | 36 (25.9) | 18 (27.3) | 16 (23.5) | 23 (32.4) | 20 (28.2) |
| therapy, n (%) | | | | | | |
| Bevacizumab | 0 | 4 (2.9) | 0 | 2 (2.9) | 0 | 2 (2.8) |
| Carboplatin | 7 (5.1) | 5 (3.6) | 4 (6.1) | 4 (5.9) | 3 (4.2) | 1 (1.4) |
| Cisplatin | 1 (0.7) | 1 (0.7) | 0 | 0 | 1 (1.4) | 1 (1.4) |
| Crizotinib | 0 | 2 (1.4) | 0 | 1 (1.5) | 0 | 1 (1.4) |
| Docetaxel | 15 (10.9) | 9 (6.5) | 11 (16.7) | 9 (13.2) | 4 (5.6) | 0 |
| Erlotinib | 0 | 1 (0.7) | 0 | 0 | 0 | 1 (1.4) |
| Etoposide | 1 (0.7) | 1 (0.7) | 0 | 0 | 1 (1.4) | 1 (1.4) |
| Gemcitabine | 12 (8.8) | 9 (6.5) | 3 (4.5) | 3 (4.4) | 9 (12.7) | 6 (8.5) |
| Nab-Paclitaxel | 0 | 1 (0.7) | 0 | 1 (1.5) | 0 | 0 |
| Nivolumab | 3 (2.2) | 5 (3.6) | 2 (3.0) | 1 (1.5) | 1 (1.4) | 4 (5.6) |
| Other | 10 (7.3) | 15 (10.8) | 1 (1.5) | 8 (11.8) | 9 (12.7) | 7 (9.9) |
| Paclitaxel | 4 (2.9) | 1 (0.7) | 2 (3.0) | 1 (1.5) | 2 (2.8) | 0 |
| Pembrolizumab | 3 (2.2) | 1 (0.7) | 1 (1.5) | 0 | 2 (2.8) | 1 (1.4) |
| Pemetrexed | 3 (2.2) | 5 (3.6) | 1 (1.5) | 2 (2.9) | 2 (2.8) | 3 (4.2) |
| Vinorelbine | 4 (2.9) | 4 (2.9) | 0 | 1 (1.5) | 4 (5.6) | 3 (4.2) |

TABLE 9
All Grade 3-5 Adverse Events

| | TTFields Therapy | | | Immune | | | TTFields | | | | | |
|---|---|---|---|---|---|---|---|---|---|---|---|---|
| | with an Immune | | | Checkpoint | | | Therapy | | | | | |
| | Checkpoint Inhibitor | | | Inhibitor | | | with Docetaxel | | | Docetaxel | | |
| Preferred | N = 67 | | | N = 66 | | | N = 66 | | | N = 68 | | |
| term - | Grade | Grade | Grade | Grade | Grade | Grade | Grade | Grade | Grade | Grade | Grade | Grade |
| no. (%) | 3 | 4 | 5 | 3 | 4 | 5 | 3 | 4 | 5 | 3 | 4 | 5 |
| Any adverse | 29 (43.3) | 3 (4.5) | 5 (7.5) | 20 (30.3) | 6 (9.1) | 6 (9.1) | 22 (33.3) | 11 (16.7) | 8 (12.1) | 23 (33.8) | 16 (23.5) | 4 (5.9) |
| event | | | | | | | | | | | | |
| Adverse | | | | | | | | | | | | |
| events | | | | | | | | | | | | |
| Blood and | | | | | | | | | | | | |
| lymphatic | | | | | | | | | | | | |
| system | | | | | | | | | | | | |
| disorders | | | | | | | | | | | | |
| Anemia | 4 (6.0) | 1 (1.5) | 0 | 2 (3.0) | 0 | 0 | 5 (7.6) | 0 | 0 | 8 (11.8) | 0 | 0 |
| Neutropenia | 1 (1.5) | 1 (1.5) | 0 | 0 | 1 (1.5) | 0 | 3 (4.5) | 4 (6.1) | 0 | 1 (1.5) | 10 (14.7) | 0 |
| Febrile | 0 | 0 | 0 | 0 | 0 | 0 | 6 (9.1) | 0 | 0 | 3 (4.4) | 0 | 0 |
| neutropenia | | | | | | | | | | | | |
| Leukopenia | 1 (1.5) | 0 | 0 | 0 | 0 | 0 | 1 (1.5) | 0 | 0 | 3 (4.4) | 0 | 0 |
| Thrombo- | 0 | 0 | 0 | 0 | 0 | 0 | 2 (3.0) | 0 | 0 | 0 | 1 (1.5) | 0 |
| cytopenia | | | | | | | | | | | | |
| Lymphopenia | 2 (3.0) | 0 | 0 | 0 | 0 | 0 | 0 | 0 | 0 | 0 | 0 | 0 |
| Anemia | 0 | 0 | 0 | 0 | 0 | 0 | 0 | 0 | 0 | 1 (1.5) | 0 | 0 |
| macrocytic | | | | | | | | | | | | |
| Cardiac | | | | | | | | | | | | |
| disorders | | | | | | | | | | | | |
| Pericardial | 1 (1.5) | 0 | 0 | 0 | 0 | 0 | 0 | 1 (1.5) | 0 | 0 | 0 | 0 |
| effusion | | | | | | | | | | | | |
| Acute | 0 | 0 | 0 | 0 | 0 | 0 | 1 (1.5) | 0 | 0 | 0 | 0 | 0 |
| myocardial | | | | | | | | | | | | |
| infarction | | | | | | | | | | | | |
| Atrial | 0 | 0 | 0 | 0 | 0 | 0 | 0 | 0 | 0 | 1 (1.5) | 0 | 0 |
| tachycardia | | | | | | | | | | | | |
| Cardiac | 0 | 0 | 0 | 0 | 0 | 0 | 0 | 1 (1.5) | 0 | | | |
| arrest | | | | | | | | | | | | |
| Cardiac | 0 | 0 | 0 | 0 | 0 | 0 | 1 (1.5) | 0 | 0 | 0 | 0 | 0 |
| failure | | | | | | | | | | | | |
| congestive | | | | | | | | | | | | |
| Cardiac | 0 | 0 | 0 | 0 | 0 | 0 | 0 | 1 (1.5) | 0 | 0 | 0 | 0 |
| tamponade | | | | | | | | | | | | |
| Cardiac | 0 | 0 | 0 | 0 | 0 | 0 | 1 (1.5) | 0 | 0 | 0 | 0 | 0 |
| ventricular | | | | | | | | | | | | |
| thrombosis | | | | | | | | | | | | |
| Coronary | 0 | 0 | 0 | 0 | 0 | 0 | 0 | 0 | 1 (1.5) | 0 | 0 | 0 |
| artery | | | | | | | | | | | | |
| disease | | | | | | | | | | | | |
| Myocardial | 0 | 0 | 0 | 0 | 0 | 0 | 0 | 0 | 0 | 1 (1.5) | 0 | 0 |
| infarction | | | | | | | | | | | | |
| Right | 0 | 0 | 0 | 0 | 0 | 0 | 0 | 0 | 0 | 1 (1.5) | 0 | 0 |
| ventricular | | | | | | | | | | | | |
| failure | | | | | | | | | | | | |
| Endocrine | | | | | | | | | | | | |
| disorders | | | | | | | | | | | | |
| Thyroiditis | 1 (1.5) | 0 | 0 | 0 | 0 | 0 | 0 | 0 | 0 | 0 | 0 | 0 |
| subacute | | | | | | | | | | | | |
| Eye | | | | | | | | | | | | |
| disorders | | | | | | | | | | | | |
| Cataract | 0 | 0 | 0 | 0 | 0 | 0 | 1 (1.5) | 0 | 0 | 0 | 0 | 0 |
| Gastrointestinal | | | | | | | | | | | | |
| disorders | | | | | | | | | | | | |
| Diarrhea | 1 (1.5) | 0 | 0 | 0 | 0 | 0 | 1 (1.5) | 0 | 0 | 0 | 0 | 0 |
| Nausea | 0 | 0 | 0 | 1 (1.5) | 0 | 0 | 0 | 0 | 0 | 0 | 0 | 0 |
| Vomiting | 0 | 0 | 0 | 1 (1.5) | 0 | 0 | 1 (1.5) | 0 | 0 | 0 | 0 | 0 |
| Mouth | 0 | 0 | 0 | 0 | 0 | 0 | 1 (1.5) | 0 | 0 | 0 | 0 | 0 |
| ulceration | | | | | | | | | | | | |
| Stomatitis | 0 | 0 | 0 | 0 | 0 | 0 | 1 (1.5) | 0 | 0 | 0 | 0 | 0 |
| Hematochezia | 0 | 0 | 0 | 0 | 0 | 0 | 1 (1.5) | 0 | 0 | 0 | 0 | 0 |
| Ascites | 0 | 0 | 0 | 0 | 0 | 0 | 1 (1.5) | 0 | 0 | 0 | 0 | 0 |
| Autoimmune | 1 (1.5) | 0 | 0 | 0 | 0 | 0 | 0 | 0 | 0 | 0 | 0 | 0 |
| colitis | | | | | | | | | | | | |
| Ileus | 0 | 0 | 0 | 0 | 0 | 0 | 0 | 0 | 0 | 1 (1.5) | 0 | 0 |
| Intestinal | 0 | 0 | 0 | 0 | 0 | 0 | 0 | 0 | 0 | 1 (1.5) | 0 | 0 |
| dilatation | | | | | | | | | | | | |
| Intestinal | 0 | 0 | 0 | 0 | 0 | 0 | 0 | 0 | 0 | 1 (1.5) | 0 | 0 |
| hemorrhage | | | | | | | | | | | | |
| Intestinal | 0 | 0 | 1 (1.5) | 0 | 0 | 0 | 0 | 0 | 0 | 0 | 0 | 0 |
| perforation | | | | | | | | | | | | |
| Large | 0 | 0 | 0 | 0 | 0 | 0 | 0 | 1 (1.5) | 0 | 0 | 0 | 0 |
| intestine | | | | | | | | | | | | |
| perforation | | | | | | | | | | | | |
| Esophagitis | 0 | 0 | 0 | 0 | 0 | 0 | 1 (1.5) | 0 | 0 | 0 | 0 | 0 |
| Pancreatic | 0 | 0 | 0 | 0 | 0 | 0 | 0 | 0 | 0 | 1 (1.5) | 0 | 0 |
| failure | | | | | | | | | | | | |
| Pancreatitis | 0 | 0 | 0 | 1 (1.5) | 0 | 0 | 0 | 0 | 0 | 0 | 0 | 0 |
| Subileus | 1 (1.5) | 0 | 0 | 0 | 0 | 0 | 0 | 0 | 0 | 0 | 0 | 0 |
| General | | | | | | | | | | | | |
| disorders and | | | | | | | | | | | | |
| administration | | | | | | | | | | | | |
| site | | | | | | | | | | | | |
| conditions | | | | | | | | | | | | |
| Fatigue | 1 (1.5) | 0 | 0 | 1 (1.5) | 0 | 0 | 2 (3.0) | 0 | 0 | 4 (5.9) | 0 | 0 |
| Asthenia | 1 (1.5) | 0 | 0 | 1 (1.5) | 0 | 0 | 2 (3.0) | 0 | 0 | 4 (5.9) | 0 | 0 |
| Oedema | 0 | 0 | 0 | 0 | 0 | 0 | 0 | 0 | 0 | 2 (2.9) | 0 | 0 |
| peripheral | | | | | | | | | | | | |
| Pyrexia | 0 | 0 | 0 | 0 | 0 | 0 | 1 (1.5) | 0 | 0 | 0 | 0 | 0 |
| General | 1 (1.5) | 0 | 0 | 1 (1.5) | 1 (1.5) | 0 | 4 (6.1) | 0 | 0 | 0 | 0 | 0 |
| physical | | | | | | | | | | | | |
| health | | | | | | | | | | | | |
| deterioration | | | | | | | | | | | | |
| Non-cardiac | 0 | 0 | 0 | 0 | 0 | 0 | 1 (1.5) | 0 | 0 | 1 (1.5) | 0 | 0 |
| chest pain | | | | | | | | | | | | |
| Localized | 0 | 0 | 0 | 0 | 0 | 0 | 1 (1.5) | 0 | 0 | 0 | 0 | 0 |
| oedema | | | | | | | | | | | | |
| Chest pain | 1 (1.5) | 0 | 0 | 0 | 0 | 0 | 0 | 0 | 0 | 0 | 0 | 0 |
| Mucosal | 0 | 0 | 0 | 0 | 0 | 0 | 0 | 0 | 0 | 1 (1.5) | 0 | 0 |
| inflammation | | | | | | | | | | | | |
| Euthanasia | 0 | 0 | 0 | | 0 | 0 | 0 | 0 | 0 | 0 | 0 | 1 (1.5) |
| | | | | 0 | | | | | | | | |
| Multiple | 0 | 0 | 0 | 0 | 0 | 0 | 1 (1.5) | 0 | 0 | 0 | 0 | 0 |
| organ | | | | | | | | | | | | |
| dysfunction | | | | | | | | | | | | |
| syndrome | | | | | | | | | | | | |
| Hepatobiliary | | | | | | | | | | | | |
| disorders | | | | | | | | | | | | |
| Cholelithiasis | 0 | 0 | 0 | 1 (1.5) | 0 | 0 | 0 | 0 | 0 | 0 | 0 | 0 |
| Cholangitis | 0 | 0 | 0 | 0 | 0 | 0 | 0 | 0 | 0 | 1 (1.5) | 0 | 0 |
| Cholecystitis | 0 | 0 | 0 | 1 (1.5) | 0 | 0 | 0 | 0 | 0 | 0 | 0 | 0 |
| Liver | 0 | 0 | 0 | 0 | 0 | 0 | 0 | 0 | 0 | 1 (1.5) | 0 | 0 |
| disorder | | | | | | | | | | | | |
| Immune | | | | | | | | | | | | |
| system | | | | | | | | | | | | |
| disorders | | | | | | | | | | | | |
| Drug | 0 | 0 | 0 | 1 (1.5) | 0 | 0 | 0 | 0 | 0 | 1 (1.5) | 0 | 0 |
| hypersensitivity | | | | | | | | | | | | |
| Infections and | | | | | | | | | | | | |
| infestations | | | | | | | | | | | | |
| Pneumonia | 4 (6.0) | 0 | 0 | 3 (4.5) | 0 | 1 (1.5) | 7 (10.6) | 2 (3.0) | 0 | 7 (10.3) | 0 | 0 |
| Urinary | 1 (1.5) | 0 | 0 | 1 (1.5) | 0 | 0 | 2 (3.0) | 0 | 0 | 0 | 0 | 0 |
| tract | | | | | | | | | | | | |
| infection | | | | | | | | | | | | |
| Respiratory | 0 | 0 | 0 | 0 | 0 | 0 | 0 | 0 | 1 (1.5) | 0 | 0 | 0 |
| tract | | | | | | | | | | | | |
| infection | | | | | | | | | | | | |
| Upper | 0 | 0 | 0 | 0 | 0 | 0 | 1 (1.5) | 0 | 0 | 0 | 0 | 0 |
| respiratory | | | | | | | | | | | | |
| tract | | | | | | | | | | | | |
| infection | | | | | | | | | | | | |
| Bronchitis | 1 (1.5) | 0 | 0 | 0 | 0 | 0 | 0 | 0 | 0 | 0 | 0 | 0 |
| Herpes | 0 | 0 | 0 | 1 (1.5) | 0 | 0 | 0 | 0 | 0 | 0 | 0 | 0 |
| zoster | | | | | | | | | | | | |
| Sepsis | 0 | 0 | 0 | 0 | 1 (1.5) | 1 (1.5) | 0 | 2 (3.0) | 0 | 1 (1.5) | 1 (1.5) | 0 |
| Skin | 0 | 0 | 0 | 0 | 0 | 0 | 1 (1.5) | 0 | 0 | 0 | 0 | 0 |
| infection | | | | | | | | | | | | |
| COVID-19 | 0 | 0 | 0 | 0 | 1 (1.5) | 1 (1.5) | 1 (1.5) | 0 | 0 | 0 | 0 | 0 |
| pneumonia | | | | | | | | | | | | |
| Oral | 0 | 0 | 0 | 0 | 0 | 0 | 1 (1.5) | 0 | 0 | 0 | 0 | 0 |
| candidiasis | | | | | | | | | | | | |
| Septic | 0 | 1 (1.5) | 0 | 0 | 0 | 0 | 0 | 1 (1.5) | 0 | 0 | 0 | 0 |
| shock | | | | | | | | | | | | |
| Aspergillus | 0 | 0 | 0 | 0 | 0 | 0 | 0 | 0 | 1 (1.5) | 0 | 0 | 0 |
| infection | | | | | | | | | | | | |
| Bronchiolitis | 1 (1.5) | 0 | 0 | 0 | 0 | 0 | 0 | 0 | 0 | 0 | 0 | 0 |
| Clostridium | 0 | 0 | 0 | 0 | 0 | 0 | 1 (1.5) | 0 | 0 | 0 | 0 | 0 |
| bacteremia | | | | | | | | | | | | |
| Diverticulitis | 0 | 0 | 0 | 0 | 0 | 0 | 1 (1.5) | 0 | 0 | 0 | 0 | 0 |
| Encephalitis | 1 (1.5) | 0 | 0 | 0 | 0 | 0 | 0 | 0 | 0 | 0 | 0 | 0 |
| Gastroenteritis | 0 | 0 | 0 | 0 | 0 | 0 | 0 | 0 | 0 | 1 (1.5) | 0 | 0 |
| norovirus | | | | | | | | | | | | |
| Gastroenteritis | 0 | 0 | 0 | 0 | 0 | 0 | 1 (1.5) | 0 | 0 | 0 | 0 | 0 |
| salmonella | | | | | | | | | | | | |
| Infection | 0 | 0 | 0 | 0 | 0 | 0 | 1 (1.5) | 0 | 0 | 0 | 0 | 0 |
| Lung | 0 | 0 | 0 | 1 (1.5) | 0 | 0 | 0 | 0 | 0 | 0 | 0 | 0 |
| abscess | | | | | | | | | | | | |
| Pneumonia | 0 | 0 | 0 | 0 | 0 | 0 | 0 | 0 | 0 | 0 | 1 (1.5) | 0 |
| bacterial | | | | | | | | | | | | |
| Pneumonia | 0 | 0 | 0 | 0 | 0 | 0 | 1 (1.5) | 0 | 0 | 0 | 0 | 0 |
| pseudomonal | | | | | | | | | | | | |
| Pneumonia | 0 | 0 | 0 | 0 | 1 (1.5) | 0 | 0 | 0 | 0 | 0 | 0 | 0 |
| staphylococcal | | | | | | | | | | | | |
| Pyelonephritis | 1 (1.5) | 0 | 0 | 0 | 0 | 0 | 0 | 0 | 0 | 0 | 0 | 0 |
| Pyopneumothorax | 0 | 0 | 0 | 0 | 0 | 0 | 0 | 0 | 0 | 1 (1.5) | 0 | 0 |
| Urosepsis | 0 | 0 | 0 | 1 (1.5) | 0 | 0 | 0 | 0 | 0 | 0 | 0 | 0 |
| Injury, | | | | | | | | | | | | |
| poisoning and | | | | | | | | | | | | |
| procedural | | | | | | | | | | | | |
| complications | | | | | | | | | | | | |
| Femoral | 0 | 0 | 0 | 0 | 0 | 0 | 0 | 1 (1.5) | 0 | 0 | 0 | 0 |
| neck | | | | | | | | | | | | |
| fracture | | | | | | | | | | | | |
| Hip fracture | 1 (1.5) | 0 | 0 | 0 | 0 | 0 | 0 | 0 | 0 | 0 | 0 | 0 |
| Investigations | | | | | | | | | | | | |
| Weight | 1 (1.5) | 0 | 0 | 0 | 0 | 0 | 0 | 0 | 0 | 0 | 0 | 0 |
| decreased | | | | | | | | | | | | |
| White blood | 0 | 0 | 0 | 0 | 1 (1.5) | 0 | 6 (9.1) | 1 (1.5) | 0 | 5 (7.4) | 2 (2.9) | 0 |
| cell count | | | | | | | | | | | | |
| decreased | | | | | | | | | | | | |
| Neutrophil | 0 | 0 | 0 | 0 | 1 (1.5) | 0 | 2 (3.0) | 5 (7.6) | 0 | 2 (2.9) | 4 (5.9) | 0 |
| count | | | | | | | | | | | | |
| decreased | | | | | | | | | | | | |
| Alanine | 0 | 0 | 0 | 2 (3.0) | 0 | 0 | 0 | 0 | 0 | 1 (1.5) | 0 | 0 |
| aminotransferase | | | | | | | | | | | | |
| increased | | | | | | | | | | | | |
| Blood | 0 | 0 | 0 | 1 (1.5) | 0 | 0 | 0 | 0 | 0 | 0 | 0 | 0 |
| bilirubin | | | | | | | | | | | | |
| increased | | | | | | | | | | | | |
| Platelet | 1 (1.5) | 0 | 0 | 0 | 0 | 0 | 0 | 0 | 0 | 0 | 0 | 0 |
| count | | | | | | | | | | | | |
| decreased | | | | | | | | | | | | |
| C-reactive | 0 | 0 | 0 | 0 | 0 | 0 | 1 (1.5) | 0 | 0 | 0 | 0 | 0 |
| protein | | | | | | | | | | | | |
| increased | | | | | | | | | | | | |
| Lymphocyte | 0 | 0 | 0 | 0 | 0 | 0 | 1 (1.5) | 0 | 0 | 0 | 0 | 0 |
| count | | | | | | | | | | | | |
| decreased | | | | | | | | | | | | |
| Hepatic | 0 | 0 | 0 | 1 (1.5) | 0 | 0 | 0 | 0 | 0 | 0 | 0 | 0 |
| enzyme | | | | | | | | | | | | |
| increased | | | | | | | | | | | | |
| Forced | 0 | 0 | 0 | 0 | 0 | 0 | 0 | 0 | 0 | 1 (1.5) | 0 | 0 |
| expiratory | | | | | | | | | | | | |
| volume | | | | | | | | | | | | |
| decreased | | | | | | | | | | | | |
| Metabolism and | | | | | | | | | | | | |
| nutrition | | | | | | | | | | | | |
| disorders | | | | | | | | | | | | |
| Decreased | 0 | 0 | 0 | 0 | 0 | 0 | 1 (1.5) | 0 | 0 | 0 | 0 | 0 |
| appetite | | | | | | | | | | | | |
| Hypoalbu- | 0 | 0 | 0 | 0 | 0 | 0 | 1 (1.5) | 0 | 0 | 1 (1.5) | 0 | 0 |
| minemia | | | | | | | | | | | | |
| Hyponatremia | 0 | 1 (1.5) | 0 | 3 (4.5) | 0 | 0 | 2 (3.0) | 0 | 0 | 2 (2.9) | 0 | 0 |
| Hypokalemia | 1 (1.5) | 0 | 0 | 0 | 0 | 0 | 2 (3.0) | 0 | 0 | 0 | 0 | 0 |
| Hyperglycemia | 2 (3.0) | 0 | 0 | 1 (1.5) | 0 | 0 | 0 | 0 | 0 | 1 (1.5) | 0 | 0 |
| Dehydration | 0 | 0 | 0 | 0 | 0 | 0 | 1 (1.5) | 0 | 0 | 1 (1.5) | 0 | 0 |
| Hypercalcemia | 0 | 0 | 0 | 1 (1.5) | 0 | 0 | 1 (1.5) | 0 | 0 | 0 | 0 | 0 |
| Hyperkalaemia | 1 (1.5) | 0 | 0 | 0 | 0 | 0 | 0 | 0 | 0 | 0 | 0 | 0 |
| Hypocalcaemia | 1 (1.5) | 0 | 0 | 0 | 0 | 0 | 0 | 0 | 0 | 0 | 0 | 0 |
| Hypertriglyc- | 0 | 0 | 0 | 1 (1.5) | 0 | 0 | 0 | 0 | 0 | 0 | 0 | 0 |
| eridaemia | | | | | | | | | | | | |
| Malnutrition | 0 | 0 | 0 | 0 | 0 | 0 | 1 (1.5) | 0 | 0 | 0 | 0 | 0 |
| Refeeding | 0 | 0 | 0 | 0 | 0 | 0 | 0 | 0 | 0 | 1 (1.5) | 0 | 0 |
| syndrome | | | | | | | | | | | | |
| Musculoskeletal | | | | | | | | | | | | |
| and | | | | | | | | | | | | |
| connective | | | | | | | | | | | | |
| tissue | | | | | | | | | | | | |
| disorders | | | | | | | | | | | | |
| Back pain | 0 | 0 | 0 | 3 (4.5) | 0 | 0 | 0 | 0 | 0 | 0 | 0 | 0 |
| Arthralgia | 1 (1.5) | 0 | 0 | 0 | 0 | 0 | 1 (1.5) | 0 | 0 | 0 | 0 | 0 |
| Myalgia | 0 | 0 | 0 | 0 | 0 | 0 | 1 (1.5) | 0 | 0 | 0 | 0 | 0 |
| Muscular | 0 | 0 | 0 | 0 | 0 | 0 | 1 (1.5) | 0 | 0 | 1 (1.5) | 0 | 0 |
| weakness | | | | | | | | | | | | |
| Bone pain | 0 | 0 | 0 | 0 | 0 | 0 | 1 (1.5) | 0 | 0 | 1 (1.5) | 0 | 0 |
| Neck pain | 0 | 0 | 0 | 0 | 0 | 0 | 0 | 0 | 0 | 1 (1.5) | 0 | 0 |
| Neoplasms | | | | | | | | | | | | |
| benign, | | | | | | | | | | | | |
| malignant and | | | | | | | | | | | | |
| unspecified | | | | | | | | | | | | |
| (incl cysts | | | | | | | | | | | | |
| and polyps) | | | | | | | | | | | | |
| Metastases | 0 | 0 | 0 | 0 | 0 | 0 | 2 (3.0) | 0 | 0 | 2 (2.9) | 0 | 0 |
| to central | | | | | | | | | | | | |
| nervous | | | | | | | | | | | | |
| system | | | | | | | | | | | | |
| Cancer pain | 0 | 0 | 0 | 1 (1.5) | 0 | 0 | 1 (1.5) | 0 | 0 | 1 (1.5) | 0 | 0 |
| Metastases | 1 (1.5) | 0 | 0 | 0 | 0 | 0 | 1 (1.5) | 0 | 0 | 0 | 0 | 0 |
| to bone | | | | | | | | | | | | |
| Tumor pain | 0 | 0 | 0 | 0 | 0 | 0 | 1 (1.5) | 0 | 0 | 0 | 0 | 0 |
| Adenocarcinoma | 1 (1.5) | 0 | 0 | 0 | 0 | 0 | 0 | 0 | 0 | 0 | 0 | |
| of colon | | | | | | | | | | | | |
| Malignant | 0 | 0 | 0 | 0 | 0 | 0 | 0 | 0 | 0 | 1 (1.5) | 0 | 0 |
| ascites | | | | | | | | | | | | |
| Malignant | 0 | 0 | 0 | 0 | 0 | 0 | 0 | 0 | 0 | 1 (1.5) | 0 | 0 |
| pleural | | | | | | | | | | | | |
| effusion | | | | | | | | | | | | |
| Metastases | 0 | 0 | 0 | 0 | 0 | 0 | 1 (1.5) | 0 | 0 | 0 | 0 | 0 |
| to meninges | | | | | | | | | | | | |
| Metastases | 1 (1.5) | 0 | 0 | 0 | 0 | 0 | 0 | 0 | 0 | 0 | 0 | |
| to skin | | | | | | | | | | | | |
| Nervous | | | | | | | | | | | | |
| system | | | | | | | | | | | | |
| disorders | | | | | | | | | | | | |
| Neuropathy | 1 (1.5) | 0 | 0 | 0 | 0 | 0 | 0 | 0 | 0 | 0 | 0 | |
| peripheral | | | | | | | | | | | | |
| Peripheral | 0 | 0 | 0 | 0 | 0 | 0 | 1 (1.5) | 0 | 0 | 1 (1.5) | 0 | 0 |
| sensory | | | | | | | | | | | | |
| neuropathy | | | | | | | | | | | | |
| Syncope | 0 | 0 | 0 | 0 | 0 | 0 | 1 (1.5) | 0 | 0 | 2 (2.9) | 0 | 0 |
| Neurotoxicity | 1 (1.5) | 0 | 0 | 0 | 0 | 0 | 0 | 0 | 0 | 0 | 0 | |
| Peripheral | 0 | 0 | 0 | 1 (1.5) | 0 | 0 | 0 | 0 | 0 | 2 (2.9) | 0 | 0 |
| sensorimotor | | | | | | | | | | | | |
| neuropathy | | | | | | | | | | | | |
| Brain oedema | 0 | 0 | 0 | 0 | 0 | 0 | 0 | 0 | 0 | 0 | 1 (1.5) | 0 |
| Cauda equina | 0 | 0 | 0 | 0 | 0 | 0 | 1 (1.5) | 0 | 0 | 0 | 0 | 0 |
| syndrome | | | | | | | | | | | | |
| Dysarthria | 0 | 0 | 0 | 0 | 0 | 0 | 1 (1.5) | 0 | 0 | 0 | 0 | 0 |
| Embolic | 1 (1.5) | 0 | 0 | 0 | 0 | 0 | 0 | 0 | 0 | 0 | 0 | |
| stroke | | | | | | | | | | | | |
| Hemiparesis | 0 | 0 | 0 | 0 | 0 | 0 | 0 | 0 | 0 | 1 (1.5) | 0 | 0 |
| Motor | 0 | 0 | 0 | 0 | 0 | 0 | 1 (1.5) | 0 | 0 | 0 | 0 | 0 |
| dysfunction | | | | | | | | | | | | |
| Nervous | 0 | 0 | 0 | 1 (1.5) | 0 | 0 | 0 | 0 | 0 | 0 | 0 | 0 |
| system | | | | | | | | | | | | |
| disorder | | | | | | | | | | | | |
| Neurologic | 0 | 0 | 0 | 0 | 0 | 0 | 0 | 0 | 0 | 1 (1.5) | 0 | 0 |
| neglect | | | | | | | | | | | | |
| syndrome | | | | | | | | | | | | |
| Quadriparesis | 0 | 1 (1.5) | 0 | 0 | 0 | 0 | 0 | 0 | 0 | 0 | 0 | 0 |
| Seizure | 0 | 0 | 0 | 0 | 0 | 0 | 1 (1.5) | 0 | 0 | 0 | 0 | 0 |
| Vocal cord | 1 (1.5) | 0 | 0 | 0 | 0 | 0 | 0 | 0 | 0 | 0 | 0 | 0 |
| paralysis | | | | | | | | | | | | |
| Psychiatric | | | | | | | | | | | | |
| disorders | | | | | | | | | | | | |
| Anxiety | 0 | 0 | 0 | 0 | 0 | 0 | 1 (1.5) | 0 | 0 | 0 | 0 | 0 |
| Confusional | 0 | 0 | 0 | 0 | 0 | 0 | 1 (1.5) | 0 | 0 | 0 | 0 | 0 |
| state | | | | | | | | | | | | |
| Mental | 0 | 0 | 0 | 0 | 0 | 0 | 0 | 0 | 0 | 0 | 1 (1.5) | 0 |
| status | | | | | | | | | | | | |
| changes | | | | | | | | | | | | |
| Renal and | | | | | | | | | | | | |
| urinary | | | | | | | | | | | | |
| disorders | | | | | | | | | | | | |
| Urinary | 0 | 0 | 0 | 0 | 0 | 0 | 1 (1.5) | 0 | 0 | 0 | 0 | 0 |
| retention | | | | | | | | | | | | |
| Acute | 0 | 0 | 0 | 0 | 0 | 0 | 1 (1.5) | 0 | 0 | 0 | 0 | 0 |
| kidney | | | | | | | | | | | | |
| injury | | | | | | | | | | | | |
| Respiratory, | | | | | | | | | | | | |
| thoracic, and | | | | | | | | | | | | |
| mediastinal | | | | | | | | | | | | |
| disorders | | | | | | | | | | | | |
| Dyspnoea | 2 (3.0) | 0 | 0 | 1 (1.5) | 0 | 0 | 7 (10.6) | 0 | 0 | 2 (2.9) | 1 (1.5) | 0 |
| Cough | 0 | 0 | 0 | 1 (1.5) | 0 | 0 | 0 | 0 | 0 | 0 | 0 | 0 |
| Hemoptysis | 2 (3.0) | 0 | 0 | 0 | 0 | 0 | 0 | 0 | 2 (3.0) | 1 (1.5) | 0 | 0 |
| Pleural | 1 (1.5) | 0 | 0 | 2 (3.0) | 0 | 0 | 2 (3.0) | 0 | 0 | 4 (5.9) | 0 | 0 |
| effusion | | | | | | | | | | | | |
| Pulmonary | 1 (1.5) | 0 | 1 (1.5) | 2 (3.0) | 1 (1.5) | 0 | 3 (4.5) | 0 | 0 | 1 (1.5) | 0 | 0 |
| embolism | | | | | | | | | | | | |
| Pneumonitis | 1 (1.5) | 0 | 0 | 2 (3.0) | 0 | 0 | 0 | 1 (1.5) | 0 | 1 (1.5) | 0 | 0 |
| Chronic | 1 (1.5) | 0 | 0 | 1 (1.5) | 0 | 1 (1.5) | 1 (1.5) | 0 | 0 | 1 (1.5) | 0 | 0 |
| obstructive | | | | | | | | | | | | |
| pulmonary | | | | | | | | | | | | |
| disease | | | | | | | | | | | | |
| Acute | 0 | 0 | 1 (1.5) | 0 | 0 | 0 | 2 (3.0) | 0 | 1 (1.5) | 0 | 0 | 2 (2.9) |
| respiratory | | | | | | | | | | | | |
| failure | | | | | | | | | | | | |
| Hypoxia | 0 | 0 | 0 | 0 | 0 | 0 | 1 (1.5) | 0 | 0 | 2 (2.9) | 0 | 0 |
| Respiratory | 0 | 0 | 0 | 0 | 1 (1.5) | 2 (3.0) | 0 | 0 | 1 (1.5) | 0 | 0 | 1 (1.5) |
| failure | | | | | | | | | | | | |
| Atelectasis | 1 (1.5) | 0 | 0 | 0 | 0 | 0 | 0 | 0 | 0 | 2 (2.9) | 0 | 0 |
| Pulmonary | 0 | 0 | 2 (3.0) | 0 | 0 | 0 | 0 | 0 | 1 (1.5) | 0 | 0 | 0 |
| hemorrhage | | | | | | | | | | | | |
| Interstitial | 0 | 0 | 0 | 1 (1.5) | 0 | 0 | 0 | 0 | 0 | 0 | 0 | 0 |
| lung disease | | | | | | | | | | | | |
| Bronchial | 0 | 0 | 0 | 0 | 0 | 0 | 0 | 0 | 0 | 0 | 2 (2.9) | 0 |
| obstruction | | | | | | | | | | | | |
| Bronchopleural | 1 (1.5) | 0 | 0 | 0 | 0 | 0 | 0 | 0 | 0 | 0 | 0 | 0 |
| fistula | | | | | | | | | | | | |
| Esophago- | 1 (1.5) | 0 | 0 | 0 | 0 | 0 | 0 | 0 | 0 | 0 | 0 | 0 |
| bronchial | | | | | | | | | | | | |
| fistula | | | | | | | | | | | | |
| Pneumonia | 0 | 0 | 0 | 0 | 0 | 0 | 1 (1.5) | 0 | 0 | 0 | 0 | 0 |
| aspiration | | | | | | | | | | | | |
| Skin and | | | | | | | | | | | | |
| subcutaneous | | | | | | | | | | | | |
| tissue | | | | | | | | | | | | |
| disorders | | | | | | | | | | | | |
| Alopecia | 0 | 0 | 0 | 0 | 0 | 0 | 0 | 0 | 0 | 1 (1.5) | 0 | 0 |
| Pruritus | 0 | 0 | 0 | 0 | 0 | 0 | 1 (1.5) | 0 | 0 | 0 | 0 | 0 |
| Rash | 1 (1.5) | 0 | 0 | 0 | 0 | 0 | 0 | 0 | 0 | 0 | 0 | 0 |
| Rash | 0 | 0 | 0 | 0 | 0 | 0 | 1 (1.5) | 0 | 0 | 0 | 0 | 0 |
| maculopapular | | | | | | | | | | | | |
| Skin | 0 | 0 | 0 | 0 | 0 | 0 | 1 (1.5) | 0 | 0 | 0 | 0 | 0 |
| toxicity | | | | | | | | | | | | |
| Rash | 0 | 0 | 0 | 1 (1.5) | 0 | 0 | 0 | 0 | 0 | 0 | 0 | 0 |
| erythematous | | | | | | | | | | | | |
| Dermatitis | 1 (1.5) | 0 | 0 | 0 | 0 | 0 | 0 | 0 | 0 | 0 | 0 | 0 |
| exfoliative | | | | | | | | | | | | |
| Skin erosion | 0 | 0 | 0 | 0 | 0 | 0 | 1 (1.5) | 0 | 0 | 0 | 0 | 0 |
| Skin reaction | 0 | 0 | 0 | 0 | 0 | 0 | 1 (1.5) | 0 | 0 | 0 | 0 | 0 |
| Vascular | | | | | | | | | | | | |
| disorders | | | | | | | | | | | | |
| Hypertension | 0 | 0 | 0 | 0 | 0 | 0 | 3 (4.5) | 0 | 0 | 0 | 0 | 0 |
| Deep vein | 1 (1.5) | 0 | 0 | 1 (1.5) | 0 | 0 | 0 | 0 | 0 | 0 | 0 | 0 |
| thrombosis | | | | | | | | | | | | |
| Embolism | 0 | 0 | 0 | 1 (1.5) | 0 | 0 | 0 | 0 | 0 | 0 | 0 | 0 |
| Jugular vein | 0 | 0 | 0 | 0 | 0 | 0 | 0 | 0 | 0 | 0 | 1 (1.5) | 0 |
| thrombosis | | | | | | | | | | | | |
| Peripheral | 1 (1.5) | 0 | 0 | 0 | 0 | 0 | 0 | 0 | 0 | 0 | 0 | 0 |
| arterial | | | | | | | | | | | | |
| occlusive | | | | | | | | | | | | |
| disease | | | | | | | | | | | | |
| Vena cava | 0 | 0 | 0 | 0 | 0 | 0 | 1 (1.5) | 0 | 0 | 0 | 0 | 0 |
| embolism | | | | | | | | | | | | |

TABLE 10
TTFields Device-Related Adverse Events

| | TTFields therapy with | | TTFields | |
|---|---|---|---|---|
| | an immune checkpoint | | therapy with | |
| | inhibitor | | docetaxel | |
| | N = 67 | | N = 66 | |
| Preferred term, n (%) | All grades | Grade 3* | All grades | Grade 3* |
| Any TTFields | 49 (73.1) | 3 (4.5) | 46 (69.7) | 5 (7.6) |
| device-related adverse | | | | |
| event | | | | |
| Serious TTFields | 1 (1.5) | | 3 (4.5) | |
| device-related adverse | | | | |
| event | | | | |
| TTFields device-related | 0 | 0 | 0 | 0 |
| adverse event | | | | |
| leading to discontinuation | | | | |
| TTFields device-related | 0 | | 0 | |
| adverse event | | | | |
| leading to death | | | | |
| TTFields device-related | | | | |
| adverse events^{†} | | | | |
| General disorders | | | | |
| and administration site | | | | |
| conditions | | | | |
| Chest pain | 1 (1.5) | 1 (1.5) | 0 | 0 |
| Infections and infestations | | | | |
| Skin infection | 0 | 0 | 2 (3.0) | 1 (1.5) |
| Respiratory, thoracic, | | | | |
| and mediastinal | | | | |
| disorders | | | | |
| Bronchopleural fistula | 1 (1.5) | 1 (1.5) | 0 | 0 |
| Skin and subcutaneous | | | | |
| tissue disorders | | | | |
| Dermatitis | 9 (13.4) | 0 | 7 (10.6) | 0 |
| Skin irritation | 9 (13.4) | 0 | 6 (9.1) | 0 |
| Pruritus | 7 (10.4) | 0 | 6 (9.1) | 1 (1.5) |
| Rash | 7 (10.4) | 0 | 4 (6.1) | 0 |
| Contact dermatitis | 5 (7.5) | 0 | 5 (7.6) | 0 |
| Maculo-papular rash | 1 (1.5) | 0 | 7 (10.6) | 0 |
| Skin toxicity | 6 (9.0) | 0 | 2 (3.0) | 1 (1.5) |
| Dermatitis exfoliative | 2 (3.0) | 1 (1.5) | 1 (1.5) | 0 |
| generalized | | | | |
| Skin erosion | 0 | 0 | 2 (3.0) | 1 (1.5) |
| Skin reaction | 0 | 0 | 2 (3.0) | 1 (1.5) |

Adverse events are compiled from the safety population.
*There were no TTFields device-related grade 4 or 5 adverse events.
^{†}5% of patients in any group and all grade 3-5 adverse events.

### B. Example 2 Tumor Treating Fields (TTFields) Therapy with Standard of Care (SOC) in Metastatic Non-Small Cell Lung Cancer (mNSCLC) Following Platinum Failure; Randomized, Phase 3 LUNAR Study

### 1. Background

TTFields are electric fields that disrupt processes critical for cancer cell viability. TTFields are delivered by a noninvasive portable device that is FDA approved for glioblastoma and mesothelioma. Preclinical NSCLC studies demonstrated that TTFields enhance the antitumor immune response, through disruption of mitosis and subsequent induction of immunogenic cell death. In addition, TTFields were shown to synergize with taxanes and immune checkpoint inhibitors (ICIs). The global, randomized, phase 3 LUNAR study assessed TTFields therapy with SOC (investigator's choice ICI or docetaxel [DTX]) for previously treated mNSCLC.

### 2. Methods:

Adults with mNSCLC progressing on or after platinum therapy (prior ICI permitted) and ECOG PS≤2 were randomized 1:1 to TTFields+SOC or SOC. TTFields therapy (150 kHz) was delivered continuously until progression or intolerable toxicity. Primary endpoint was overall survival (OS). Key secondary endpoints were OS in ICI and DTX subgroups. Other secondary endpoints included progression-free survival (PFS) and adverse events (AEs).

### 3. Results:

276 patients were randomized between February 2017 and November 2021 to receive TTFields+SOC (n=137) vs SOC (n=139). Baseline characteristics were balanced: median (m) age 64 years (range, 22-86); 65% male; 56% non-squamous; 96% ECOG PS 0-1; 89% one prior line of systemic therapy; 31% prior ICI. OS was significantly extended with TTFields+SOC vs SOC. After a minimum follow-up of 12 months (mo), mOS (95% CI) was 13.2 (10.3-15.5) mo with TTFields+SOC vs 10.0 (8.2-12.2) mo with SOC (HR 0.74; 95% CI 0.56-0.98; P=0.037). 1-year survival rates (95% CI) were 53% (44-61) and 42% (34-50), respectively (P=0.040). mPFS was 4.8 (4.1-5.7) mo and 4.1 (3.0-4.7) mo (HR 0.87; 95% CI 0.67-1.14), respectively. In patients receiving an ICI (n=134), TTFields significantly improved OS vs ICI alone: mOS (95% CI) 18.5 (10.6-30.3) vs 10.6 (8.2-17.6) mo (HR 0.63; 95% CI 0.41-0.96; P=0.032). In the DTX subgroup (n=142), patients who received TTFields had a numerically higher mOS vs DTX alone: 11.1 (95% CI 8.2-14.1) mo vs 8.9 (95% CI 6.5-12.2) mo (HR 0.87; 95% CI 0.60-1.26). The rate of AEs was similar between groups (97% TTFields+SOC vs 91% SOC). The incidence of TTFields-related AEs was 71%; majority were grade 1 and 2 local skin irritation; 8 patients (6%) reported a grade 3 AE. There were no grade 4 toxicities and no deaths attributable to TTFields.

### 4. Conclusion:

TTFields therapy significantly extended OS in patients with mNSCLC following platinum failure without exacerbating systemic toxicities, and with few high-grade device-related AEs. The efficacy and safety demonstrated in this phase 3 study warrant inclusion of TTFields therapy as part of second line SOC in mNSCLC.

### C. Example 3 (LUNAR4): Tumor Treating Fields in Pretreated Non-Small Cell Lung Cancer

### 1. Methods

i. Study Design and Oversight

LUNAR4 will be a prospective, randomized, study of TTFields therapy concomitant with checkpoint inhibitors in patients with NSCLC progressing on or after checkpoint inhibitor therapy alone or checkpoint inhibitor+chemotherapy, wherein the chemotherapy may comprise platinum-based chemotherapy.

The LUNAR4 study will be designed by the sponsor (Novocure GmbH) and the investigators. Data will be collected by the investigators and analyzed by sponsor-employed statisticians.

TTFields are expected to re-establish adaptive immunity in the setting of relapse after ICI therapy. Therefore, while retreatment of PD-1/PD-L1 inhibitor monotherapy is not recommended, concomitant therapy of TTFields with pembrolizumab may result in an improved prognosis

### II. Patient Population

Eligible participants may be adults with a histological or cytological diagnosis of NSCLC that had shown radiological progression while on or after checkpoint inhibitor therapy alone or checkpoint inhibitor+chemotherapy. Treatment with TTFields plus the checkpoint inhibitor pembrolizumab provides particular improved results for patients with a diagnosis of metastatic NSCLC that had shown progression while on or after previous treatment with a checkpoint inhibitor (PD-1/PD-L1 inhibitor) and platinum-based chemotherapy.

iii. Procedures

Eligible patients will be randomly assigned 1:1 (within 28 days of providing informed consent) to receive TTFields therapy to the thorax concomitant with investigator's choice of checkpoint inhibitor. Randomization will be determined centrally using variable blocked randomization and an IxRS system, and stratified by tumor histology, treatment (checkpoint inhibitor), and region (North America, Western Europe/Israel, and Eastern Europe).

TTFields (150 kHz) will be delivered continuously for an average of at least 18 hours per day with the NovoTTF device system (FIG. 3C). Array layouts will be determined by the investigator and modified as needed throughout the treatment period, per computed tomography scans and provided clinical practice guidelines. Patients will be trained by the investigator, other healthcare provider, or a Device Support Specialist (DSS; sponsor provided) to use the device. Arrays will be replaced (and shifted by approximately 2 cm from the original position to minimize skin irritation) every 3-4 days. TTFields usage time (device-captured data) will be reported monthly to investigators, presented as a percentage.

Follow-up visits will be conducted every 6 weeks (±1 week) for radiological assessment of disease per immune-related (ir)RECIST for patients receiving an immune checkpoint inhibitor.

TTFields therapy will be continued until progression in the thorax and/or liver or intolerable toxicity. Treatment breaks of up to 3 weeks will be allowed for TTFields-related adverse events. Patients could continue to receive TTFields therapy, even if systemic therapy is discontinued due to progression outside of the thorax/liver or intolerable toxicity.

Messenger RNA (mRNA) expression profiling in archival material (biopsy specimens, peripheral blood) may be completed to assess gene expression and attempt to define a gene set critical for clinical response to TTFields therapy concomitant with pembrolizumab. TTFields concomitant with pembrolizumab may induce responses in tumors that reflect an inflamed/immune phenotype based on gene expression signatures capturing PD-L1 and interferon-gamma transcriptional programs. Global profiling may also be pursued. Expression of individual genes related to the immune system may also be evaluated such as immune signatures and critical cytokines (e.g., IL-10).

Tissue and blood derivatives can be subjected to proteomic profiling studies using a variety of platforms that could include but are not limited to immunoassay, flow cytometry, Liquid Chromatography/Mass Spectrometry, to identify novel protein biomarker(s) that may aid in patient selection for TTFields therapy concomitant with pembrolizumab

The application of new technologies, such as next generation sequencing, has provided scientists the opportunity to define certain tumor types at the genetic level as being ‘hypermutated’ or can detect the presence of specific T-cell clones within the tumor microenvironment or in the peripheral blood. The hypermutated state and/or increased T-cell clonality may correlate with response to TTFields therapy concomitant with pembrolizumab, and/or that the converse, ‘hypomutated’ state or lack of dominant T-cell clones may correlate with non-response. Genetic variation within a clinical study population may therefore correlate with response to the treatment under evaluation.

iv. Study Endpoints

A primary endpoint will be overall survival in patients receiving TTFields with checkpoint inhibitor compared to checkpoint inhibitor therapy alone. Other secondary endpoints will include progression-free survival and overall response rate (both per radiological assessment), overall survival and progression-free survival by squamous and non-squamous histology, and adverse events.

Overall survival will be defined as the time from randomization to date-of-death from any cause or censoring at the last follow-up date. Progression-free survival will be defined as the time from the date of randomization until the date of disease progression in the liver or thorax, or death by any cause. Deaths that occur without the investigator detecting tumor progression will be considered progression events if they occurred within 6 weeks of the final tumor assessment prior to death. Otherwise, the patient will be censored at the date of the final tumor assessment. Patients who had not progressed or died at the time of analysis will be censored at the date of the most recent evaluable tumor assessment. Patients with no post-baseline tumor assessment will be censored at the date of randomization. Overall radiological response rate will be defined as a complete or partial response, and best response (categorized by complete response, partial response, stable disease, progressive disease, or not evaluable) will be calculated for each treatment.

v. Statistical Analysis

Overall survival and progression-free survival will be evaluated with two-sided log rank tests, at an alpha level of 0.05, stratified by treatment (, and tumor histology. Medians, confidence intervals (CIs), and rates will be estimated using the Kaplan-Meier method. Hazard ratios with 95% CI and P-values will be estimated using stratified Cox proportional hazards models with stratification variables introduced as covariates. For comparison of overall response rates, the 95% CI will be calculated based on the exact binomial distribution (Clopper-Pearson). The difference between treatment arms, as well as the associated two-sided 95% CI, will be calculated using a chi square test with significance at 0.05 (two-sided P value). Best responses will be summarized per treatment group.

Efficacy endpoints will be analyzed in all randomized patients (the intent-to-treat population). Safety and treatment data will be compiled from all patients who received any study therapy and will be analyzed according to the actual treatment received. Analyses were performed using SAS software, version 9.4 (SAS Institute).

### Aspects Forming Part of the Description

1. A checkpoint inhibitor for use in a method of increasing survival of a subject having cancer, the method comprising:
a. applying an alternating electric field to a target site of the subject for a period of time, the alternating electric field having a frequency and field strength, wherein the target site comprises one or more cancer cells, and
b. administering a therapeutically effective amount of the checkpoint inhibitor to the subject.

2. A checkpoint inhibitor for use in a method of treating a subject having cancer, the method comprising:
a. applying an alternating electric field to a target site of the subject for a period of time, the alternating electric field having a frequency and field strength, wherein the target site comprises one or more cancer cells, and
b. administering a therapeutically effective amount of a checkpoint inhibitor to the subject.

3. A method of increasing survival of a subject having cancer comprising:
a. applying an alternating electric field to a target site of the subject for a period of time, the alternating electric field having a frequency and field strength, wherein the target site comprises one or more cancer cells, and
b. administering a therapeutically effective amount of a checkpoint inhibitor to the subject.
4. A method of treating a subject having cancer comprising:
a. applying an alternating electric field to a target site of the subject for a period of time, the alternating electric field having a frequency and field strength, wherein the target site comprises one or more cancer cells, and
b. administering a therapeutically effective amount of a checkpoint inhibitor to the subject.

5. The checkpoint inhibitor for use of aspects 1-2, or the method of aspects 3-4, wherein the checkpoint inhibitor is Nivolumab, Pembrolizumab, or Atezolizumab.

6. The checkpoint inhibitor for use of any one of aspects 1, 2, and 5, or the method of any one of aspects 2-5, wherein the cancer is metastatic non-small cell lung cancer.

7. The checkpoint inhibitor for use of any one of aspects 1, 2, 5, and 6, or the method of any one of aspects 1-6, wherein the subject was previously treated for non-small cell lung cancer with systemic therapy.

8. The checkpoint inhibitor for use of any one of aspects 1, 2, 5, 6, and 7 or the method of any one of aspects 1-7, wherein the systemic therapy did not comprise treatment with a checkpoint inhibitor.

9. The checkpoint inhibitor for use of any one of aspects 1, 2, 5, 6, 7, and 8 or the method of any one of aspects 1-7, wherein the systemic therapy comprised treatment with a checkpoint inhibitor.

10. The checkpoint inhibitor for use of any one of aspects 1, 2, 5, 6, 7, 8, and 9 or the method of any one of aspects 1-7, wherein the systemic therapy comprised platinum-based-chemotherapy.

11. The checkpoint inhibitor for use of any one of aspects 1, 2, 5, 6, 7, 8, 9, and 10 or the method of any one of aspects 1-7, wherein the systemic therapy comprised chemotherapy and treatment with a checkpoint inhibitor.

12. The checkpoint inhibitor for use of any one of aspects 1, 2, 5-11 or the method of any one of aspects 1-11, wherein chemotherapy comprised platinum-based-chemotherapy.

13. The checkpoint inhibitor for use of any one of aspects 1, 2, 5-12 or the method of any one of aspects 7-12, wherein the systemic therapy comprised treatment with a checkpoint inhibitor.

14. The checkpoint inhibitor for use of any one of aspects 1, 2, 5-13 or the method of any one of aspects 7-13, wherein the non-small cell lung cancer in the subject progressed on or after the previous systemic treatment.

15. The checkpoint inhibitor for use of any one of aspects 1, 2, 5-14, or the method of any one of aspects 1-14 3, wherein the increased survival is increased progression free survival, optionally wherein:
the increased survival is compared to survival of a subject administered the same therapeutically effective amount of checkpoint inhibitor but without applying alternating electric field;
the increased survival is an increased survival of at least 1 month compared to a subject administered the same therapeutically effective amount of checkpoint inhibitor but without applying alternating electric field;
the cancer is a squamous cell NSCLC and the increased survival is an increased survival of at least 3 months compared to a subject administered the same therapeutically effective amount of checkpoint inhibitor but without applying alternating electric field; or
the cancer is a non-squamous cell NSCLC and the increased survival is an increased survival of at least 2 months compared to a subject administered the same therapeutically effective amount of checkpoint inhibitor but without applying alternating electric field.

16. The checkpoint inhibitor for use of any one of aspects 1, 2, and 5-15, or the method of any one of aspects 3-15, wherein the frequency of the alternating electric field is between 100 kHz and 1 MHz.

17. The checkpoint inhibitor for use of any one of aspects 1, 2, and 5-16, or the method of any one of aspects 3-16, wherein the frequency of the alternating electric field is 150 kHz.

18. The checkpoint inhibitor for use of any one of aspects 1-2, or the method of any one of aspects 3-4, wherein the cancer is metastatic non-small cell lung cancer, wherein the subject was previously treated for non-small cell lung cancer with systemic therapy including platinum-based-chemotherapy, and wherein the non-small cell lung cancer in the subject progressed on or after the previous systemic treatment, wherein the frequency of the alternating electric field is 150 kHz.

19. The checkpoint inhibitor for use of any one of aspects 1-2, or the method of any one of aspects 3-4, wherein the cancer is metastatic non-small cell lung cancer, wherein the subject was previously treated for non-small cell lung cancer with systemic therapy including treatment with a checkpoint inhibitor and platinum-based-chemotherapy, and wherein the non-small cell lung cancer in the subject progressed on or after the previous systemic treatment,
wherein the frequency of the alternating electric field is 150 kHz, and
wherein the checkpoint inhibitor administered in the step of administering the therapeutically effective amount of the checkpoint inhibitor to the subject is Pembrolizumab.

20. The checkpoint inhibitor for use of any one of aspects 1, 2, and 5-19 or the method of any one of aspects 1-19, wherein the checkpoint inhibitor of the systemic therapy was a PD-1/PD-L1 inhibitor.

21. The checkpoint inhibitor for use of any one of aspects 1, 2, and 5-20, or the method of any one of aspects 3-20, wherein applying the alternating electric field to the target site of the subject for the period of time comprises applying the electric field to the target site continuously for an average of at least 18 hours per day.

22. The checkpoint inhibitor for use of any one of aspects 1, 2, and 5-21, or the method of any one of aspects 3-21, wherein applying the alternating electric field to the target site of the subject for the period of time comprises applying the electric field to the target site continuously until progression of the cancer or until intolerable toxicity.

23. The checkpoint inhibitor for use of any one of aspects, 1, 2, and 5-22, or the method of any one of aspects 3-22, wherein the field strength of the alternating electric field is produced by generating the alternating electric field with an applied voltage of at least 50 V RMS, optionally wherein the field strength of the alternating electric field is produced by generating the alternating electric field with an applied voltage of between 10-250 V RMS.

24. The checkpoint inhibitor for use of any one of aspects, 1, 2, and 5-23, or the method of any one of aspects 3-23, further comprising administering docetaxel.

25. Docetaxel for use in a method of increasing survival of a subject having cancer, the method comprising:
a. applying an alternating electric field to a target site of the subject for a period of time, the alternating electric field having a frequency and field strength, wherein the target site comprises one or more cancer cells, and
b. administering a therapeutically effective amount of the docetaxel to the subject.

26. Docetaxel for use in a method of treating a subject having cancer, the method comprising:
a. applying an alternating electric field to a target site of the subject for a period of time, the alternating electric field having a frequency and field strength, wherein the target site comprises one or more cancer cells, and
b. administering a therapeutically effective amount of the docetaxel to the subject.

27. A method of increasing survival of a subject having cancer comprising:
a. applying an alternating electric field to a target site of the subject for a period of time, the alternating electric field having a frequency and field strength, wherein the target site comprises one or more cancer cells, and
b. administering a therapeutically effective amount of docetaxel to the subject.

28. A method of treating a subject having cancer comprising:
a. applying an alternating electric field to a target site of the subject for a period of time, the alternating electric field having a frequency and field strength, wherein the target site comprises one or more cancer cells, and
b. administering a therapeutically effective amount of docetaxel to the subject.

29. The docetaxel for use of any one of aspects 25-26, or the method of any one of aspects 27-28, wherein the cancer is metastatic non-small cell lung cancer.

30. The docetaxel for use of any one of aspects 25-26, or 29 or the method of any one of aspects 27-29, wherein the subject was previously treated for non-small cell lung cancer with systemic therapy.

31. The docetaxel for use of any one of aspects 25-26, 29-30 or the method of any one of aspects 27-30, wherein the systemic therapy did not comprise treatment with a checkpoint inhibitor.

32. The docetaxel for use of any one of aspects 25-26, or 29-30 or the method of any one of aspects 27-30, wherein the systemic therapy comprised treatment with a checkpoint inhibitor.

33. The docetaxel for use of any one of aspects 25-26 or 29-30 or the method of any one of aspects 27-30, wherein the systemic therapy comprised platinum-based-chemotherapy.

34. The docetaxel for use or the method of aspect 30, wherein the systemic therapy comprised chemotherapy and treatment with a checkpoint inhibitor.

35. The docetaxel for use of any one of aspects 25-26 or 29-34 or the method of any one of aspects 27-34, wherein chemotherapy comprised platinum-based-chemotherapy.

36. The docetaxel for use of any one of aspects 25-26 or 29-35 or the method of any one of aspects 27-35, wherein the systemic therapy comprised treatment with a checkpoint inhibitor.

37. The docetaxel for use of any one of aspects 25-26 or 29-36 or the method of any one of aspects 27-36, wherein the non-small cell lung cancer in the subject progressed on or after the previous systemic treatment.

38. The docetaxel for use of aspects 25-26 or 29-37, or the method of any one of aspects 27-37, wherein the increased survival is increased progression free survival.

39. The docetaxel for use of any one of aspects 25, 26, and 29-38, or the method of any one of aspects 27-38, wherein the frequency of the alternating electric field is between 100 kHz and 1 MHz.

40. The docetaxel for use 25-26 or 29-39 or the method of any one of aspects 27-39, wherein the frequency of the alternating electric field is 150 kHz.

41. The docetaxel for use of any one of aspects 25-26 or 29-40, or the method of any one of aspects 27-40, wherein the cancer is metastatic non-small cell lung cancer, wherein the subject was previously treated for non-small cell lung cancer with systemic therapy including platinum-based-chemotherapy, and wherein the non-small cell lung cancer in the subject progressed on or after the previous systemic treatment, wherein the frequency of the alternating electric field is 150 kHz.

42. The docetaxel for use 25-26 or 29-41 or the method of any one of aspects 27-41, wherein the checkpoint inhibitor of the systemic therapy was a PD-1/PD-L1 inhibitor.

43. The docetaxel for use of any one of aspects 25, 26, and 29-42, or the method of any one of aspects 27-42, wherein applying the alternating electric field to the target site of the subject for the period of time comprises applying the electric field to the target site continuously for an average of at least 18 hours per day.

44. The docetaxel for use of any one of aspects 25, 26, and 29-43, or the method of any one of aspects 27-43, wherein applying the alternating electric field to the target site of the subject for the period of time comprises applying the electric field to the target site continuously until progression of the cancer or until intolerable toxicity.

45. The docetaxel for use of any one of aspects 25, 26, and 29-44, or the method of any one of aspects 27-44, wherein the field strength of the alternating electric field is produced by generating the alternating electric field with an applied voltage of at least 50 V RMS, optionally wherein the field strength of the alternating electric field is produced by generating the alternating electric field with an applied voltage of between 10-250 V RMS.46. A checkpoint inhibitor for use in a method of increasing survival of a subject having non-small cell lung cancer, the method comprising:
a. applying an alternating electric field to a target site of the subject for a period of time, the alternating electric field having a frequency and field strength, wherein the target site comprises one or more non-small cell lung cancer cells, and
b. administering a therapeutically effective amount of the checkpoint inhibitor to the subject.

47. A checkpoint inhibitor for use in a method of treating a subject having non-small cell lung cancer, the method comprising:
a. applying an alternating electric field to a target site of the subject for a period of time, the alternating electric field having a frequency and field strength, wherein the target site comprises one or more non-small cell lung cancer cells, and
b. administering a therapeutically effective amount of the checkpoint inhibitor to the subject.

48. A method of increasing survival of a subject having non-small cell lung cancer comprising:
a. applying an alternating electric field to a target site of the subject for a period of time, the alternating electric field having a frequency and field strength, wherein the target site comprises one or more non-small cell lung cancer cells, and
b. administering a therapeutically effective amount of a checkpoint inhibitor to the subject.

49. A method of treating a subject having non-small cell lung cancer comprising:
a. applying an alternating electric field to a target site of the subject for a period of time, the alternating electric field having a frequency and field strength, wherein the target site comprises one or more non-small cell lung cancer cells, and
b. administering a therapeutically effective amount of a checkpoint inhibitor to the subject.

50. The checkpoint inhibitor for use of any one of aspects 46-47, or the method of aspects any one of aspects 18-49, wherein the checkpoint inhibitor is a PD-1 inhibitor or PDL-1 inhibitor.

51. The checkpoint inhibitor for use of any one of aspects 46-47, or the method of aspects any one of aspects 18-49, wherein the checkpoint inhibitor is ipilimumab (Yervoy), pembrolizumab (Keytruda), nivolumab (Opdivo), cemiplimab (trade name Libtayo), and dostarlimab (Jemperli), atezolizumab (Tecentriq), durvalumab (Imfinzi), or avelumab (Bavencio).

52. The checkpoint inhibitor for use of aspect 46 or the method of aspect 48, wherein the increased survival is increased progression free survival.

53. The checkpoint inhibitor for use of any one of aspects 46, 47, and 50-52, or the method of any one of aspects 48-52, wherein the frequency of the alternating electric field is between 100 kHz and 1 MHz.

54. The checkpoint inhibitor for use of any one of aspects 46, 47, and 50-53, or the method of any one of aspects 48-52, further comprising administering a chemotherapy to the subject.

55. The checkpoint inhibitor for use of any one of aspects 46, 47, and 50-54, or the method of any of aspects 48-53, further comprising discontinuing the alternating electrical field.

56. The checkpoint inhibitor for use of any one of aspects 46, 47, and 50-55, or the method of any of aspects 48 to 57, wherein the subject was previously treated with a checkpoint inhibitor.

57. The checkpoint inhibitor for use of any one of aspects 46, 47, and 50-56, or the method of any of aspects 48-56, wherein the subject was previously treated with a checkpoint inhibitor and chemotherapy.

58. The checkpoint inhibitor for use of any one of aspects 46, 47, and 50-56 or the method of aspect 48-56, wherein the non-small cell lung cancer in the subject progressed on or after the previous treatment with the checkpoint inhibitor.

59. The checkpoint inhibitor for use of any one of aspects 46, 47, and 50-58 or the method of any one of aspects 48-57, wherein the non-small cell lung cancer in the subject progressed on or after the previous treatment with the checkpoint inhibitor and the chemotherapy.

60. The checkpoint inhibitor for use of any one of aspects 46, 47, and 50-59 or the method of any one of aspects 56 or 58, wherein the subject has been diagnosed with radiological progression of the non-small cell lung cancer on or after the previous treatment with the checkpoint inhibitor.

61. The checkpoint inhibitor for use of any one of aspects 46, 47, and 50-60 or the method of any one of aspects 57 or 58, wherein the subject has been diagnosed with radiological progression of the non-small cell lung cancer on or after the previous treatment with the checkpoint inhibitor and chemotherapy.

62. The checkpoint inhibitor for use of any one of aspects 46, 47, and 50-61 or the method of any one of aspects 56, 58 or 60, wherein the subject progressed to metastatic disease within 6 months of completing the previous treatment with the checkpoint inhibitor.

63. The checkpoint inhibitor for use or the method of any one of aspects 57, 59 or 61, wherein the subject progressed to metastatic disease within 6 months of completing the previous treatment with the checkpoint inhibitor and chemotherapy.

64. The checkpoint inhibitor for use of any one of aspects 57, 59, 61, or 63 or the method of any one of aspects 57, 59, 61, or 63, wherein the chemotherapy was a platinum-based chemotherapy.

65. The checkpoint inhibitor for use of any one of aspects 47, 48, and 50-55, or the method of any one of aspects 48-55, wherein the subject was previously treated with a checkpoint inhibitor and was not previously treated with a non-platinum-based chemotherapy or a platinum-based chemotherapy.

66. The checkpoint inhibitor for use of any one of aspects 46, 47, and 50-55, or the method of any one of aspects 8-55, wherein the subject was previously treated with a checkpoint inhibitor was not previously treated with a platinum-based chemotherapy.

67. The checkpoint inhibitor for use of any one of aspects 46, 47, and 50-66 or the method of any one of aspect 48-66, wherein the non-small cell lung cancer in the subject progressed on or after the previous treatment with the checkpoint inhibitor.

68. The checkpoint inhibitor for use 46, 47, and 50-67 or the method of any one of aspects

48-67, wherein the subject has been diagnosed with radiological progression of the non-small cell lung cancer on or after the previous treatment with the checkpoint inhibitor.

69. The checkpoint inhibitor for use of any one of aspects 46, 47, and 50-55, or the method of any one of aspects 48-55, wherein the cancer has progressed on or after treatment with a platinum-based systemic therapy.

70. The checkpoint inhibitor for use any one of aspects 46, 47, and 50-69 or the method of aspect 48-69, wherein the subject has been diagnosed with radiological progression while on or after platinum-based systemic therapy.

71. The checkpoint inhibitor for use any one of aspects 46, 47, and 50-70 or the method of any one of aspects 48-70, wherein the platinum-based systemic therapy comprised one or more of nedaplatin, satraplatin, cisplatin, carboplatin, and oxaliplatin.

72. The checkpoint inhibitor for use any one of aspects 46, 47, and 50-71 or the method of any one of aspects 48-71, wherein the subject progressed to metastatic disease within 6 months of completing a platinum-based systemic therapy.

73. The checkpoint inhibitor for use of any one of aspects 46, 47, and 50-72, or the method of any one of aspects 48-72, wherein the frequency of the alternating electric field is 150 kHz.

74. The checkpoint inhibitor for use of any one of aspects 46, 47, and 50-73, or the method of any one of aspects 48-73, wherein applying the alternating electric field to the target site of the subject for the period of time comprises applying the electric field to the target site continuously for an average of at least 18 hours per day.

75. The checkpoint inhibitor for use of any one of aspects 46, 47, and 50-74, or the method of any one of aspects 48-74, wherein applying the alternating electric field to the target site of the subject for the period of time comprises applying the electric field to the target site continuously until progression of the cancer or until intolerable toxicity.

76. The checkpoint inhibitor for use of any one of aspects 46, 47, and 50-75, or the method of any one of aspects 48-71, wherein the field strength of the alternating electric field is produced by generating the alternating electric field with an applied voltage of at least 50 V RMS, optionally wherein the field strength of the alternating electric field is produced by generating the alternating electric field with an applied voltage of between 10-250 V RMS.

77. The checkpoint inhibitor for use of any one of aspects 46, 47, and 50-76, or the method of any one of aspects 48-76, the method further comprising administering a taxane.

78. The checkpoint inhibitor for use of any one of aspects 46, 47, and 50-76, or the method of any one of aspects 48-76, wherein the taxane is docetaxel, paclitaxel, cabazitaxel, abraxane, or a combination thereof.

79. The checkpoint inhibitor for use of any one of aspects 46, 47, and 50-78, or the method of any one of aspects 48-74, further comprising administering a therapeutically effective amount of a second immune checkpoint inhibitor.

80 The checkpoint inhibitor for use of any of aspects 1, 2, 5-24, 46, 47, and 50-79, the docetaxel for use of any of aspects 25, 26, and 29-45 or the method of any of aspects 4-24, 27-45 and 48-79, wherein the alternating electrical field is applied to the thorax of the subject having cancer.

81. The checkpoint inhibitor for use of any of aspects 1, 2, 5-24, 46, 47, and 50-80, the docetaxel for use of any of aspects 25, 26, 29-45, and 80 or the method of any of aspects 4-24, 27-45, and 48-80, wherein the subject has not had a cerebrovascular accident within six (6) months prior to administration of the alternating electric field.

82. The checkpoint inhibitor for use of any of aspects 1, 2, 5-24, 46, 47, and 50-81, the docetaxel for use of any of aspects 25, 26, 29-45, 80, and 81 or the method of any of aspects 4-24, 27-45, and 48-81, wherein the subject has not had a non-NSCLC malignancy within 3 years prior to administration of the alternating electric field.

83. The checkpoint inhibitor for use of any of aspects 1, 2, 5-24, 46, 47, and 50-82, the docetaxel for use of any of aspects 25, 26, 29-45, and 80-82, or the method of any one of aspects 4-24, 27-45, and 48-82, wherein the subject has not had a non-NSCLC other than stage I prostate cancer, non-melanoma skin cancer, or in situ cervical cancer or breast cancer malignancy within 3 years prior to administration of the alternating electric field.

84. The checkpoint inhibitor for use of any of aspects 1, 2, 5-24, 46, 47, and 50-83, the docetaxel for use of any of aspects 25, 26, 29-45, and 80-83 or the method of any of aspects 4-24, 27-45, and 48-83, further comprising screening the subject to determine whether the administering the therapeutically effective amount of a checkpoint inhibitor is effective in increasing survival or treating the subject.

85. The checkpoint inhibitor for use of any of aspects 1, 2, 5-24, 46, 47, and 50-84, the docetaxel for use of any of aspects 25, 26, 29-45, and 80-84 or the method of any of aspects 4-24, 27-45, and 48-84, wherein the subject does not have a clinically significant hematological, hepatic, renal, or cardiac dysfunction.

86. The checkpoint inhibitor for use of any of aspects 1, 2, 5-24, 46, 47, and 50-85, the docetaxel for use of any of aspects 25, 26, 29-45, and 80-85 or the method of any of aspects 4-24, 27-45, and 48-85, wherein the subject is a non-smoker, current smoker, or former smoker.

87. The checkpoint inhibitor for use of any of aspects 1, 2, 5-24, 46, 47, and 50-86, the docetaxel for use of any of aspects 25, 26, 29-45, and 80-86 or the method of any of aspects 4-24, 27-45, and 48-86, wherein the subject has a non-squamous cancer or squamous cancer.

88. The checkpoint inhibitor for use of any of aspects 1, 2, 5-24, 46, 47, and 50-87, the docetaxel for use of any of aspects 25, 26, 29-45, and 80-87 or the method of any of aspects 4-24, 27-45, and 48-87, wherein the subject has liver metastases.

89. The checkpoint inhibitor for use of any of aspects 1, 2, 5-24, 46, 47, and 50-88, the docetaxel for use of any of aspects 25, 26, 29-45, and 80-88 or the method of any of aspects 4-24, 27-45, and 48-88, wherein the subject having cancer is at least 22 years of age.

90. The checkpoint inhibitor for use of any of aspects 1, 2, 5-24, 46, 47, and 50-89, the docetaxel for use of any of aspects 25, 26, 29-45, and 80-89 or the method of any of aspects 4-24, 27-45, and 48-89, wherein the subject does not have a central nervous system metastases.

91. The checkpoint inhibitor for use of any of aspects 1, 2, 5-24, 46, 47, and 50-90, the docetaxel for use of any of aspects 25, 26, 29-45, and 80-90 or the method of any of aspects 4-24, 27-45, and 48-90, wherein the subject does not have a second or third degree heart blockage, uncontrolled hypertension, congestive heart failure, a history of arrhythmia that is symptomatic or requires treatment, a history of pericarditis, or a history of interstitial lung disease.

92. The checkpoint inhibitor for use of any of aspects 1, 2, 5-24, 46, 47, and 50-91, the docetaxel for use of any of aspects 25, 26, 29-45, and 80-91 or the method of any of aspects 4-24, 27-45, and 48-91, wherein the subject is not pregnant.

93. A taxane for use in a method of increasing survival of a subject having cancer comprising:
a. applying an alternating electric field to a target site of the subject for a period of time, the alternating electric field having a frequency and field strength, wherein the target site comprises one or more cancer cells, and
b. administering a therapeutically effective amount of the taxane to the subject.

94. A taxane for use in a method of treating a subject having cancer comprising:
a. applying an alternating electric field to a target site of the subject for a period of time, the alternating electric field having a frequency and field strength, wherein the target site comprises one or more cancer cells, and
b. administering a therapeutically effective amount of the taxane to the subject.

95. A method of increasing survival of a subject having cancer comprising:
a. applying an alternating electric field to a target site of the subject for a period of time, the alternating electric field having a frequency and field strength, wherein the target site comprises one or more cancer cells, and
b. administering a therapeutically effective amount of a taxane to the subject.

96. A method of treating a subject having cancer comprising:
a. applying an alternating electric field to a target site of the subject for a period of time, the alternating electric field having a frequency and field strength, wherein the target site comprises one or more cancer cells, and
b. administering a therapeutically effective amount of a taxane to the subject.

97. The taxane for use of any one of aspects 93-94, or the method of any one of aspects 95-96, wherein the taxane is docetaxel, paclitaxel, cabazitaxel, abraxane, or a combination thereof.

98. The taxane for use of any one of aspects 93-94, and 97, the method of any one of aspects 95-97, wherein the cancer is metastatic non-small cell lung cancer (NSCLC).

99. The taxane for use of any one of aspects 93-94, and 97-98, the method of aspects 95-98, wherein the increased survival is increased progression free survival.

100. The taxane for use of any one of aspects 93-94, and 97-99, the method of any one of aspects 95-99, wherein the increased survival is compared to survival of a subject administered the same therapeutically effective amount of taxene but without applying alternating electric field.

101. The taxane for use or the method of aspect 100, wherein the increased survival is an increased survival of at least 1 month compared to a subject administered the same therapeutically effective amount of taxane but without applying alternating electric field.

102. The taxane for use of any one of aspects 93-94, and 97-101, the method of any one of aspects 95-101, wherein the NSCLC is a squamous cell NSCLC.

103. The taxane for use or the method of aspect 102, wherein the increased survival is an increased survival of at least 3 months compared to a subject administered the same therapeutically effective amount of taxane but without applying alternating electric field.

104. The taxane for use of any one of aspects 93-94, and 97-101, the method of any one of aspect 95-101, wherein the NSCLC is a non-squamous cell NSCLC.

105. The taxane for use or the method of aspect 104, wherein the increased survival is an increased survival of at least 2 months compared to a subject administered the same therapeutically effective amount of taxane but without applying alternating electric field.

106. The taxane for use of any one of aspects 93-94, and 97-105, the method of any one of aspects 95-101, wherein the frequency of the alternating electric field is between 100 kHz and 1 MHz.

107. A kit, for use in any of the methods of 1-24 and 46-92, comprising:
a field generating apparatus configured to generate the alternating electric field; means for applying the alternating electric field to the subject; and the checkpoint inhibitor for use in the method.

108. A kit, for use in any of the methods of 25-45 and 80-92, comprising:
a field generating apparatus configured to generate the alternating electric field;
means for applying the alternating electric field to the subject; and
the docetaxel for use in the method.

106. A kit, for use in any of the methods of claims 93-106, comprising:
a field generating apparatus configured to generate the alternating electric field;
means for applying the alternating electric field to the subject; and
the taxane for use in the method.

1.-2. (canceled)

3. A method of increasing survival of a subject having cancer comprising:
a. applying an alternating electric field to a target site of the subject for a period of time, the alternating electric field having a frequency and field strength, wherein the target site comprises one or more cancer cells, and
b. administering a therapeutically effective amount of a checkpoint inhibitor to the subject.

4. A method of treating a subject having cancer comprising:
a. applying an alternating electric field to a target site of the subject for a period of time, the alternating electric field having a frequency and field strength, wherein the target site comprises one or more cancer cells, and
b. administering a therapeutically effective amount of a checkpoint inhibitor to the subject.

5. The method of claim 4, wherein the checkpoint inhibitor is Nivolumab, Pembrolizumab, or Atezolizumab.

6. The method of claim 4, wherein the cancer is metastatic non-small cell lung cancer.

7. The method of claim 6, wherein the subject was previously treated for non-small cell lung cancer with systemic therapy.

8. The method of claim 7, wherein the systemic therapy did not comprise treatment with a checkpoint inhibitor.

9. The method of claim 7, wherein the systemic therapy comprised treatment with a checkpoint inhibitor.

10. The method of claim 7, wherein the systemic therapy comprised platinum-based-chemotherapy.

11. The method of claim 7, wherein the systemic therapy comprised chemotherapy and treatment with a checkpoint inhibitor.

12. The method of claim 11, wherein chemotherapy comprised platinum-based-chemotherapy.

13. The method of claim 7, wherein the systemic therapy comprised treatment with a checkpoint inhibitor.

14. The method of claim 7, wherein the non-small cell lung cancer in the subject progressed on or after the previous systemic treatment.

15. (canceled)

16. The method of claim 4, wherein the frequency of the alternating electric field is between 100 kHz and 1 MHz.

17. (canceled)

18. The method claim 4, wherein the cancer is metastatic non-small cell lung cancer, wherein the subject was previously treated for non-small cell lung cancer with systemic therapy including platinum-based-chemotherapy, and wherein the non-small cell lung cancer in the subject progressed on or after the previous systemic treatment, wherein the frequency of the alternating electric field is 150 kHz.

19. The method of claim 4, wherein the cancer is metastatic non-small cell lung cancer, wherein the subject was previously treated for non-small cell lung cancer with systemic therapy including treatment with a checkpoint inhibitor and platinum-based-chemotherapy, and wherein the non-small cell lung cancer in the subject progressed on or after the previous systemic treatment,
wherein the frequency of the alternating electric field is 150 kHz, and
wherein the checkpoint inhibitor administered in the step of administering the therapeutically effective amount of the checkpoint inhibitor to the subject is Pembrolizumab.

20. The method of claim 18, wherein the checkpoint inhibitor of the systemic therapy was a PD-1/PD-L1 inhibitor.

21. The the method of claim 4, wherein applying the alternating electric field to the target site of the subject for the period of time comprises applying the electric field to the target site continuously for an average of at least 18 hours per day.

22. The method of claim 4, wherein applying the alternating electric field to the target site of the subject for the period of time comprises applying the electric field to the target site continuously until progression of the cancer or until intolerable toxicity.

23. (canceled)

24. The method of claim 4, further comprising administering docetaxel to the subject.

25.-27. (canceled)

28. A method of treating a subject having cancer comprising:
a. applying an alternating electric field to a target site of the subject for a period of time, the alternating electric field having a frequency and field strength, wherein the target site comprises one or more cancer cells, and
b. administering a therapeutically effective amount of docetaxel to the subject.

29.-92. (canceled)
